(19) United States
(12) Patent Application Publication
(10) Pub. No.: US 2009/0074787 A1
(43) Pub. Date: Mar. 19, 2009

(54) Anti-CTLA4 Antibody and Indolinone Combination Therapy for Treatment of Cancer

(75) Inventors: Jesus Gomez-Navarro, Mystic, CT (US); Charles Michael Baum, San Diego, CA (US)

Correspondence Address: PFIZER INC, 10555 SCIENCE CENTER DRIVE, SAN DIEGO, CA, 92121, US

(73) Assignee: Pfizer, Inc., Pfizer Products, Inc.

(21) Appl. No.: 11/817,395

(22) PCT Filed: Mar. 3, 2006
(86) PCT No.: PCT/US06/07651

§ 371 Date: May 23, 2008

Related U.S. Application Data
(60) Provisional application No. 60/664,653, filed on Mar. 23, 2005.

(51) Int. Cl. A61K 39/395; A61P 31/00

(52) U.S. Cl. 424/142.1; 424/174.1

(57) ABSTRACT

The invention relates to administration of an anti-CTLA4 antibody, particularly human antibodies to human CTLA4, such as those having amino acid sequences of antibodies 3.1.1, 4.1.1, 4.8.1, 4.10.2, 4.13.1, 4.14.3, 6.1.1, ticilimumab (also referred to as 11.2.1 or CP-675,206), 11.6.1, 11.7.1, 12.3.1.1, 12.9.1.1, and ipilimumab (also referred to as 10D1 or MDX-010), in combination with an indolinone receptor tyrosine kinase inhibitor (RTKI), e.g., N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide (compound 1), N-[2-(ethylamino)ethyl]-5-[(Z)-(5-fluoro-2-oxo-1,2-dihydro-3H-indol-3-ylidene)methyl]-2,4-dimethyl-1H-pyrrole-3-carboxamide (compound 2), and 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide (compound 3), for treatment of cancer. The invention relates to administering a combination of an anti-CTLA4 antibody and an indolinone RTKI such as, inter alia, compound 1. The invention relates to neoadjuvant, adjuvant, first-line, second-line, and third-line therapy of cancer, whether localized or metastasized, and at any point(s) along the disease continuum (e.g., at any stage of the cancer).

### BACKGROUND OF THE INVENTION

Cancer is now the leading cause of death in the United States. Currently, it is typically treated with one or a combination of three types of therapies: surgery, radiation, and chemotherapy. Chemotherapy involves the disruption of cell replication or cell metabolism. The adverse effects of systemic chemotherapy used in the treatment of neoplastic disease can be life threatening and have become of major importance to the clinical management of cancer patients.

The development of targeted therapies is focused on specific targeting of neoplastic cells while sparing normal tissues in order to decrease side effects. Targeted therapies have focused on angiogenesis because as tumor cells grow they require a source of nutrients. Growth factors (e.g., VEGF and PDGF) elaborated by the tumor cells stimulate angiogenesis for development of new vessels to supply key nutrients and provide a means for the tumor cells to metastasize (Klagsburn and D'Amore, 1996, Cytokine & Growth Factor Reviews 7:259-270). Further, because of their role in angiogenesis, therapeutic approaches have further focused on receptor-type tyrosine kinases (RTK) and inhibitors (RTKIs) thereof.

Cell culture and gene knockout experiments indicate that each receptor contributes to different aspects of angiogenesis. Moreover, tumor growth has been shown to be susceptible to the antiangiogenic effects of VEGF receptor antagonists (see, e.g., Kim et al., 1993, Nature 362:841-844; Weidner et al., 1991, N. Engl. J. Med. 324:1-8). In addition, PDGF plays an important role in angiogenesis by supporting the growth and survival of pericytes necessary for normal vascular development.

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer, colorectal, breast, and pancreatic cancers, and neuroendocrine tumors (NET). Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes. Therefore, the development of therapeutic methods for inhibiting, regulating and/or modulating the signal transduction of tyrosine kinases is desirable.

An alternative and/or additional approach to cancer therapy is to target the immune system (“immunotherapy”) rather than and/or in addition to targeting the tumor itself. One cancer immunotherapy approach targets cytotoxic T lymphocyte-associated antigen 4 (CTLA4; CD152), which is a cell surface receptor expressed on activated T cells. Binding of CTLA4 to its natural ligands, B7.1 (CD80) and B7.2 (CD86), delivers a negative regulatory signal to T cells, and blocking this negative signal results in enhanced T cell immune function and antitumor activity in animal models (Thompson and Allison Immunity 7:445-450 (1997); McCoy and LeGros Immunol. & Cell Biol. 77:1-10 (1999)). Several studies have demonstrated that CTLA4 blockade using antibodies markedly enhances T cell-mediated killing of tumors and can induce antitumor immunity (Leach et al., Science 271:1734-1736 (1996); Kwon et al. Proc. Natl. Acad. Sci. USA 94:8099-8103 (1997); Kwon et al., Natl. Acad. Sci. USA 96:15074-15079 (1999)).

Although use of anti-CTLA4 antibodies to induce an anti-tumor response holds great promise in the treatment of cancer, there is a long-term need to develop novel therapies to treat tumors, more particularly, solid tumors, with such antibodies. Similarly, despite the successes of currently available anti-cancer treatments, complete responses to these treatments are infrequently observed, and the patient population refractory to these treatments is still large. Thus, development of new therapeutic regimens, particularly those capable of augmenting or potentiating the anti-tumor activity of other anti-neoplastic agents, preferably, RTKIs, while reducing the cytotoxic side effects of current chemotherapeutics, is necessary. The present invention meets these needs.

### SUMMARY OF THE INVENTION

The invention includes a method for the treatment of cancer in a patient in need of such treatment. The method comprises administering to the patient a therapeutically effective amount of an anti-CTLA4 antibody, or antigen-binding portion thereof, in combination with a therapeutically effective amount of an indolinone receptor tyrosine kinase inhibitor (RTKI).

In one aspect, the indolinone RTKI is at least one compound selected from the group consisting of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide, N-[2-(ethylamino)ethyl]-5-[(Z)-(5-fluoro-2-oxo-1,2-dihydro-3H-indol-3-ylidene)methyl]-2,4-dimethyl-1H-pyrrole-3-carboxamide, and 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, or a pharmaceutically acceptable salt thereof.

In another aspect, the RTKI is N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide.

In yet another aspect, the therapeutically effective amount of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide ranges from about 25 mg to 87.5 mg per day and the N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide is administered orally.

In another aspect, the therapeutically effective amount of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide ranges from about 37.5 mg to 50 mg per day.

In another aspect, the N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide is administered according to a dosing regimen selected from the group consisting of administration every day, administration every day for approximately four weeks, administration every day for approximately four weeks followed by a resting period of about two weeks, administration every day for approximately three weeks followed by a resting period of about one week, and administration every day for approximately two weeks followed by a resting period of about one week.

In yet a further aspect, the treatment is a therapy selected from the group consisting of neoadjuvant therapy, adjuvant therapy, first-line therapy and second-line therapy.

In another aspect, the therapeutically effective amount of the human anti-CTLA4 antibody ranges from about 0.1 mg/kg to 50 mg/kg.

In a further aspect, the therapeutically effective amount of the human anti-CTLA4 antibody ranges from about 0.3 mg/kg to 20 mg/kg.

In yet another aspect, the therapeutically effective amount of the human anti-CTLA4 antibody is selected from the group consisting of at least 1 mg/kg, at least 3 mg/kg, at least 6 mg/kg, at least 10 mg/kg, and at least 15 mg/kg.

In another aspect, the antibody is administered according to a dosing regimen selected from the group consisting of administration of about 6 mg/kg every twenty-eight days, administration of about 6 mg/kg every three months, administration of about 10 mg/kg every twenty-eight days, administration of about 10 mg/kg every three months, administration of about 15 mg/kg every twenty-eight days, and administration of about 15 mg/kg every three months.

In one aspect, the N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide is administered at about 37.5 mg per day and the anti-CTLA4 antibody is administered according to a dosing regimen selected from the group consisting of administration of about 10 mg/kg every three months and administration of about 15 mg/kg every three months.

In yet a further aspect, the cancer is selected from the group consisting of breast cancer, prostate cancer, ovarian cancer, pancreatic cancer, lung cancer, acute myeloid leukemia, colorectal carcinoma, renal cell carcinoma, gastrointestinal stromal tumor, and sarcoma.

In another aspect, the anti-CTLA4 antibody, or antigen-binding portion thereof, is at least one antibody selected from the group consisting of:

(a) a human antibody having a binding affinity for CTLA4 of about 10⁻⁸ or greater, and which inhibits binding between CTLA4 and B7-1, and binding between CTLA4 and B7-2;

(b) a human antibody having an amino acid sequence comprising at least one human CDR sequence that corresponds to a CDR sequence from an antibody selected from the group consisting of 4.1.1, 4.8.1, 4.10.2, 4.13.1, 4.14.3, 6.1.1, ticilimumab (also referred to as antibody 11.2.1 or CP-675,206), 11.6.1, 11.7.1, 12.3.1.1, 12.9.1.1, and ipilimumab;

(c) a human antibody having the amino acid sequences of the heavy and light chains of an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab;

(d) an antibody, or antigen-binding portion thereof, that competes for binding with CTLA4 with at least one antibody having the amino acid sequences of the heavy and light chains of an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab; and

(e) an antibody, or antigen-binding portion thereof, that cross-competes for binding with CTLA4 with at least one antibody having the amino acid sequences of the heavy and light chains of an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab.

In one aspect, the antibody is a human antibody having the amino acid sequences of the heavy and light chains of an antibody selected from the group consisting of 4.1.1, 4.13.1, ticilimumab and ipilimumab.

Most preferably, the antibody is ticilimumab.

In another aspect, the antibody, or antigen-binding portion thereof, comprises a heavy chain and a light chain wherein the amino acid sequences of the heavy chain variable domain of the heavy chain and the light chain variable domain of the light chain are selected from the group consisting of:

(a) the amino acid sequence of SEQ ID NO:3 and the amino acid sequence of SEQ ID NO:9;

(b) the amino acid sequence of SEQ ID NO:15 and the amino acid sequence of SEQ ID NO:21;

(c) the amino acid sequence of SEQ ID NO:27 and the amino acid sequence of SEQ ID NO:33;

(d) the amino acid sequence encoded by the nucleic acid sequence of SEQ ID NO:1 and the amino acid sequence encoded by the nucleic acid sequence of SEQ ID NO:7;

(e) the amino acid sequence encoded by the nucleic acid sequence of SEQ ID NO:13 and the amino acid sequence encoded by the nucleic acid sequence of SEQ ID NO:19;

(f) the amino acid sequence encoded by the nucleic acid sequence of SEQ ID NO:25 and the amino acid sequence encoded by the nucleic acid sequence of SEQ ID NO:31; and

(g) the amino acid sequences of the heavy and light chains of ipilimumab.

In yet another aspect, the antibody, or antigen-binding portion thereof, is an antibody selected from the group consisting of:

(a) an antibody comprising the amino acid sequences set forth in SEQ ID NO:4, SEQ ID NO:5, SEQ ID NO:6, SEQ ID NO:10, SEQ ID NO:11 and SEQ ID NO:12;

(b) an antibody comprising the amino acid sequences set forth in SEQ ID NO:16, SEQ ID NO:17, SEQ ID NO:18, SEQ ID NO:22, SEQ ID NO:23 and SEQ ID NO:24; and

(c) an antibody comprising the amino acid sequences set forth in SEQ ID NO:28, SEQ ID NO:29, SEQ ID NO:30, SEQ ID NO:34, SEQ ID NO:35 and SEQ ID NO:36.

In another aspect, the antibody, or antigen-binding portion thereof, comprises a heavy chain variable region having the amino acid sequence set forth in SEQ ID NO:27 and a light chain variable region having the amino acid sequence set forth in SEQ ID NO:33.

In yet another aspect, the antibody is selected from the group consisting of:

(a) an antibody comprising the amino acid sequences set forth in SEQ ID NO:2 and SEQ ID NO:8;

(b) an antibody comprising the amino acid sequences set forth in SEQ ID NO:14 and SEQ ID NO:20; and

(c) an antibody comprising the amino acid sequences set forth in SEQ ID NO:26 and SEQ ID NO:32.

In a further aspect, the antibody is administered after completion of a course of the indolinone RTKI and most preferably, wherein the level of immune response in the patient is greater than the level of immune response in the patient during or immediately after the course of indolinone RTKI.

In one aspect, the antibody is administered from about one to one-hundred days after the course of indolinone RTKI.

The invention includes a pharmaceutical composition for treatment of cancer. The composition comprises a therapeutically effective amount of an anti-CTLA4 antibody, or antigen-binding portion thereof, and a therapeutically effective amount of an indolinone RTKI, and a pharmaceutically acceptable carrier.

In one aspect, the indolinone RTKI is at least one compound selected from the group consisting of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide, N-[2-(ethylamino)ethyl]-5-[(Z)-(5-fluoro-2-oxo-1,2-dihydro-3H-indol-3-ylidene)methyl]-2,4-dimethyl-1H-pyrrole-3-carboxamide, and 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, or a pharmaceutically acceptable salt thereof.

### BRIEF DESCRIPTION OF THE DRAWINGS

The foregoing summary, as well as the following detailed description of the invention, will be better understood when read in conjunction with the appended drawings. For the purpose of illustrating the invention there are shown in the drawings embodiment(s) which are presently preferred. It should be understood, however, that the invention is not limited to the precise arrangements and instrumentalities shown.

In the drawings:

FIG. 1, comprising FIGS. 1A-1D, shows the nucleotide and amino acid sequences of anti-CTLA4 antibody 4.1.1. FIG. 1A shows the full length nucleotide sequence for the 4.1.1 heavy chain (SEQ ID NO:1). FIG. 1B shows the full length amino acid sequence for the 4.1.1 heavy chain (SEQ ID NO:2), and the amino acid sequence for the 4.1.1 heavy chain variable region (SEQ ID NO:3) designated between brackets “[ ]”. The amino acid sequence of each 4.1.1 heavy chain CDR is underlined. The CDR sequences are as follows: CDR1: GFTFSSHGMH (SEQ ID NO:4); CDR2: VIWYDGRNKYYADSV (SEQ ID NO:5); and CDR3: GGHFGPFDY (SEQ ID NO:6). FIG. 1C shows the nucleotide sequence for the 4.1.1 light chain (SEQ ID NO:7). FIG. 1D shows the amino acid sequence of the full length 4.1.1 light chain (SEQ ID NO:8), and the variable region as indicated between brackets “[ ]” (SEQ ID NO:9). The amino acid sequence of each CDR is indicated as follows: CDR1: RASQSISSSFLA (SEQ ID NO:10); CDR2: GASSRAT (SEQ ID NO:11); and CDR3: QQYGTSPWT (SEQ ID NO:12).

FIG. 2, comprising FIGS. 2A-2D, shows the nucleotide and amino acid sequences of anti-CTLA4 antibody 4.13.1. FIG. 2A shows the full length nucleotide sequence for the 4.13.1 heavy chain (SEQ ID NO:13). FIG. 2B shows the full length amino acid sequence for the 4.13.1 heavy chain (SEQ ID NO:14), and the amino acid sequence for the 4.13.1 heavy chain variable region (SEQ ID NO:15) designated between brackets “[ ]”. The amino acid sequence of each 4.13.1 heavy chain CDR is underlined. The CDR sequences are as follows: CDR1: GFTFSSHGIH (SEQ ID NO:16); CDR2: VIWYDGRNKDYADSV (SEQ ID NO:12); and CDR3: VAPLGPLDY (SEQ ID NO:18). FIG. 2C shows the nucleotide sequence for the 4.13.1 light chain (SEQ ID NO:19). FIG. 2D shows the amino acid sequence of the full length 4.13.1 light chain (SEQ ID NO:20), and the variable region as indicated between brackets “[ ]” (SEQ ID NO:21). The amino acid sequence of each CDR is indicated as follows: CDR1: RASQSVSSYLA (SEQ ID NO:22); CDR2: GASSRAT (SEQ ID NO:23); and CDR3: QQYGRSPFT (SEQ ID NO:24).

FIG. 3, comprising FIGS. 3A-3D, shows the nucleotide and amino acid sequences of anti-CTLA4 antibody ticilimumab. FIG. 3A shows the full length nucleotide sequence for the ticilimumab heavy chain (SEQ ID NO:25). FIG. 3B shows the full length amino acid sequence for the ticilimumab heavy chain (SEQ ID NO:26), and the amino acid sequence for the ticilimumab heavy chain variable region (SEQ ID NO:27) designated between brackets “[ ]”. The amino acid sequence of each ticilimumab heavy chain CDR is underlined. The CDR sequences are as follows: CDR1: GFTFSSYGMH (SEQ ID NO:28); CDR2: VIWYDGSNKYYADSV (SEQ ID NO:29); and CDR3: DPRGATLYYYYYGMDV (SEQ ID NO:30). FIG. 3C shows the nucleotide sequence for the ticilimumab light chain (SEQ ID NO:31). FIG. 3D shows the amino acid sequence of the full length ticilimumab light chain (SEQ ID NO:32), and the variable region as indicated between brackets “[ ]” (SEQ ID NO:33). The amino acid sequence of each CDR is indicated as follows: CDR1: RASQSINSYLD (SEQ ID NO:34); CDR2: AASSLQS (SEQ ID NO:35); and CDR3: QQYYSTPFT (SEQ ID NO:36).

### DETAILED DESCRIPTION OF THE INVENTION

The invention relates to the use of anti-CTLA4 antibodies in combination with at least one indolinone (e.g., compound 1, compound 2, compound 3, and the like) to treat cancer in a patient in need of such treatment. Compound 1 or its L-malate salt is also referred to as SU11248, SU011248, sunitinib malate (USAN/WHO designation) or SUTENT™ (L-malate salt). The invention further relates to treatment of cancer by combination of the antibody-indolinone combination with at least one additional agent.

Antibodies employable in the present invention, and methods of producing them, are described in the International Application No. PCT/US99/30895, published on Jun. 29, 2000 as WO 00/37504 (e.g., ticilimumab, also known as 11.2.1 and CP-675,206), European Patent Appl. No. EP 1262193 A1, published Apr. 12, 2002, U.S. patent application Ser. No. 09/472,087, now issued as U.S. Pat. No. 6,682,736, U.S. patent application Ser. No. 09/948,939, now published as U.S. Pat. App. Pub. No. 2002/0086014 (e.g., ipilimumab, also known as 10D1 and MDX-010, Medarex, Princeton, N.J.), each of which is incorporated by reference herein. While information on the amino and nucleic acid sequences relating to these antibodies is provided herein, further information can be found in U.S. Pat. No. 6,682,736, as well as published applications WO 00/37504, EP 1262193, and US2002/0086014; the sequences set forth in those applications are hereby incorporated herein by reference.

Certain uses for these antibodies to treat various cancers were discussed in U.S. patent application Ser. No. 10/153,382, now published as U.S. Patent Application Publication No. 2003/0086930, which is incorporated by reference as if set forth in its entirety herein.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

Preferred amino acid substitutions are those which: (1) reduce susceptibility to proteolysis, (2) reduce susceptibility to oxidation, (3) alter binding affinity for forming protein complexes, and (4) confer or modify other physicochemical or functional properties of such analogs. Analogs comprising substitutions, deletions, and/or insertions can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990); Altschul et al., Nucleic Acids Res. 25:3389-402 (1997); herein incorporated by reference.

An intact “antibody” comprises at least two heavy (H) chains and two light (L) chains inter-connected by disulfide bonds. See generally, Fundamental Immunology, Ch. 7 (Paul, W., ed., 2nd ed. Raven Press, N.Y. (1989)) (incorporated by reference in its entirety for all purposes). Each heavy chain is comprised of a heavy chain variable region (HCVR or V_{H}) and a heavy chain constant region (CH). The heavy chain constant region is comprised of three domains, CH1, CH2 and CH3. Each light chain is comprised of a light chain variable region (LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antibody” can include antigen-binding portions of an intact antibody that retain capacity to specifically bind the antigen of the intact antibody, e.g., CTLA4. Antigen-binding portions may be produced by recombinant DNA techniques or by enzymatic or chemical cleavage of intact antibodies.

Examples of antigen-binding portions include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the VL and VH domains of a single arm of an antibody, (v) a single domain antibody (“dAb”), which consists of a VH domain as described in Ward et al., Nature 341:544-546 (1989); and (vi) an isolated complementarity determining region (CDR). Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); See, e.g., Bird et al. Science 242:423-426 (1988); and Huston et al. Proc. Natl. Acad. Sci. USA 85:5879-5883 (1988)). Such single chain antibodies are included by reference to the term “antibody”.

A “bispecific antibody” has two different binding specificities, see, e.g., U.S. Pat. No. 5,922,845 and U.S. Pat. No. 5,837,243; Zeilder J. Immunol. 163:1246-1252 (1999); Somasundaram Hum. Antibodies 9:47-54 (1999); Keler Cancer Res. 57:4008-4014 (1997). For example, the invention provides bispecific antibodies having one binding site for a cell surface antigen, such as human CTLA4, and a second binding site for an Fc receptor on the surface of an effector cell. The invention also provides multispecific antibodies, which have at least three binding sites.

The term “bispecific antibodies” further includes “diabodies.” Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen binding sites (See, e.g., Holliger et al., Proc. Natl. Acad. Sci. USA 90:6444-6448 (1993); Poljak et al., Structure 2:1121-1123 (1994)).

The terms “human antibody” or “human sequence antibody”, as used interchangeably herein, include antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include “chimeric” antibodies in which CDR sequences derived from the germline of another mammalian species, such as a mouse, have been grafted onto human framework sequences (i.e., “humanized” or PRIMATIZED™ antibodies).

The term “chimeric antibody” as used herein means an antibody that comprises regions from two or more different antibodies. In one embodiment, one or more of the CDRs are derived from a human anti-CTLA4 antibody. In another embodiment, all of the CDRs are derived from a human anti-CTLA4 antibody. In another embodiment, the CDRs from more than one human anti-CTLA4 antibodies are combined in a chimeric human antibody. For instance, a chimeric antibody may comprise a CDR1 from the light chain of a first human anti-CD40 antibody, a CDR2 from the light chain of a second human anti-CTLA4 antibody and a CDR3 and CDR3 from the light chain of a third human anti-CTLA4 antibody, and the CDRs from the heavy chain may be derived from one or more other anti-CD40 antibodies. Further, the framework regions may be derived from one of the same anti-CTLA4 antibodies or from one or more different human(s).

Moreover, as discussed previously herein, chimeric antibody includes an antibody comprising a portion derived from the germline sequences of more than one species.

By the term “effective amount”, or “therapeutically effective amount,” as used herein, is meant an amount that when administered to a mammal, preferably a human, mediates a detectable therapeutic response compared to the response detected in the absence of the compound. A therapeutic response, such as, but not limited to, inhibition of and/or decreased tumor growth, tumor size, metastasis, and the like, can be readily assessed by a plethora of art-recognized methods, including, e.g., such methods as disclosed herein.

The skilled artisan would understand that the effective amount of the compound or composition administered herein varies and can be readily determined based on a number of factors such as the disease or condition being treated, the stage of the disease, the age and health and physical condition of the mammal being treated, the severity of the disease, the particular compound being administered, and the like.

A “therapeutic effective amount”, or “effective amount,” is intended to qualify the amount of an agent required to detectably reduce to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 3) inhibition (i.e., slowing to some extent, preferably stopping) of tumor metastasis; 4) inhibition, to some extent, of tumor growth; 5) relieving or reducing to some extent one or more of the symptoms associated with the disorder; and/or 6) relieving or reducing the side effects associated with the administration of anticancer agents.

By the term “compete”, as used herein with regard to an antibody, is meant that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). For instance, cross-competing antibodies can bind to the epitope, or portion of the epitope, to which the antibodies of the invention (e.g., 3.1.1, 4.1.1, 4.8.1, 4.10.2, 4.13.1, 4.14.3, 6.1.1, ticilimumab, 11.6.1, 11.7.1, 12.3.1.1, and 12.9.1.1) bind. Both competing and cross-competing antibodies are encompassed by the present invention. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

By “indolinone” or “indolinone receptor tyrosine kinase inhibitor”, as the term is used herein, is meant any compound having a structure comprising a 2-indolinone core, such as, e.g., compound 1, any metabolite thereof, including, but not limited to, the compound 2, and compound 3, set forth below.

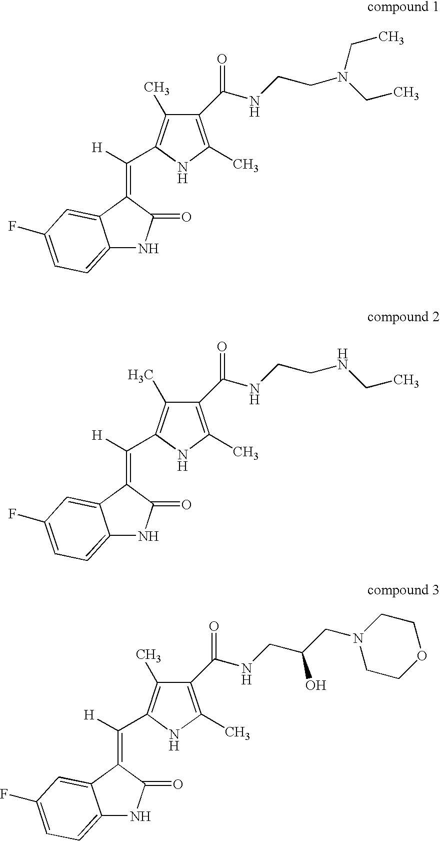

Such compounds are described in International Publication Nos. WO 2003/016305 (US 2003/0069298), WO 2005/033098 (US 2005/0118255) and WO 2004/024127 (US 2004/229229), and U.S. Pat. Nos. 6,573,293 and 6,653,308, each of which is incorporated herein in its entirety for all purposes, and wherein the compound comprises detectable inhibitory activity against any activity of at least one receptor tyrosine kinase, and any pharmaceutically acceptable salt(s) thereof. More preferably, the compound detectably inhibits KIT, FLT3, VEGFR, and/or PDGFR. Even more preferably, the compound detectably inhibits KIT, FLT3, VEGFR and PDGFR, and yet more preferably, the compound is compound 1 and its active metabolite compound 2, as well as compound 3.

“Instructional material,” as that term is used herein, includes a publication, a recording, a diagram, or any other medium of expression which can be used to communicate the usefulness of the compound, combination, and/or composition of the invention in the kit for affecting, alleviating or treating the various diseases or disorders recited herein. Optionally, or alternately, the instructional material can describe one or more methods of alleviating the diseases or disorders in a cell, a tissue, or a mammal, including as disclosed elsewhere herein.

The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively.

Except when noted, the terms “patient” or “subject” are used interchangeably and refer to mammals such as human patients and non-human primates, as well as veterinary subjects such as rabbits, rats, and mice, and other animals. Preferably, patient refers to a human.

The phrase “pharmaceutically acceptable salt(s)”, as used herein, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bistosylate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, thiethiodode, and valerate salts. Preferred salts of compounds 1-3 are disclosed in PCT Publication No. 2003/016305, U.S. patent application Ser. No. 10/956,420, filed Sep. 30, 2004, and PCT Application No. PCT/IB2004/003070, filed Sep. 20, 2004, the disclosures of which are incorporated herein by reference in their entireties. Particularly preferred salts of compound 1 include malate salts, most preferably an L-malate salt. A particularly preferred salt of compound 3 is a maleate salt.

Conventional notation is used herein to portray polypeptide sequences: the left-hand end of a polypeptide sequence is the amino-terminus; the right-hand end of a polypeptide sequence is the carboxyl-terminus.

By the phrase “specifically binds,” as used herein, is meant a compound, e.g., a protein, a nucleic acid, an antibody, and the like, which recognizes and binds a specific molecule, but does not substantially recognize or bind other molecules in a sample. For instance, an antibody or a peptide inhibitor which recognizes and binds a cognate ligand (e.g., an anti-CTLA4 antibody that binds with its cognate antigen, CTLA4) in a sample, but does not substantially recognize or bind other molecules in the sample. Thus, under designated assay conditions, the specified binding moiety (e.g., an antibody or an antigen-binding portion thereof) binds preferentially to a particular target molecule and does not bind in a significant amount to other components present in a test sample. A variety of assay formats may be used to select an antibody that specifically binds a molecule of interest. For example, solid-phase ELISA immunoassay, immunoprecipitation, BIAcore and Western blot analysis are used to identify an antibody that specifically reacts with CTLA4. Typically a specific or selective reaction will be at least twice background signal or noise and more typically more than 10 times background, even more specifically, an antibody is said to “specifically bind” an antigen when the equilibrium dissociation constant (K_{D}) is ≦1 μM, preferably ≦100 nM and most preferably ≦10 n M.

The term “K_{D}” refers to the equilibrium dissociation constant of a particular antibody-antigen interaction.

As used herein, “substantially pure” means an object species is the predominant species present (i.e., on a molar basis it is more abundant than any other individual species in the composition), and preferably a substantially purified fraction is a composition wherein the object species (e.g., an anti-CTLA4 antibody) comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

As used herein, to “treat” means reducing the frequency with which symptoms of a disease (i.e., tumor growth and/or metastasis, or other effect mediated by the numbers and/or activity of immune cells, and the like) are experienced by a patient. The term includes the administration of the compounds or agents of the present invention to prevent or delay the onset of the symptoms, complications, or biochemical indicia of a disease (e.g., elevation of PSA level), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

“Combination therapy” embraces the administration of a protein kinase inhibitor, preferably, compound 1, compound 2, and compound 3, even more preferably, compound 1, and compound 2, and most preferably, compound 1, and a CTLA4 antibody as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” embraces administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, both the therapeutic agents may be administered orally or both therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a second and different antineoplastic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

### DESCRIPTION

The invention relates to novel therapeutic methods comprising co-administering a combination of an anti-CTLA4 antibody and an indolinone, preferably, compound 1, compound 2 or compound 3, for treatment of cancer, e.g., renal cell carcinoma, breast, prostate, gastrointestinal stromal, colorectal cancer, lung, Non-Hodgkin's Lymphoma, thyroid, brain tumors, ovarian cancer, bladder cancer, hepatocellular carcinoma, cervical cancer, head and neck cancer, acute and chronic leukemias, myeloid and lymphoid leukemias, pancreatic cancer, Hodgkin's disease, melanoma, squamous cell carcinoma of the skin, Kaposi's sarcoma, sarcomas of other types (e.g., liposarcoma, osteosarcoma) among many others, in a patient in need of such treatment.

In one embodiment, the method comprises administering an indolinone RTKI in combination with the antibody. In one aspect, the method provides neoadjuvant, adjuvant, first-line, and second-line therapy for cancer. In another embodiment, the antibody-indolinone RTKI combination is administered with at least one additional therapeutic agent, such as, but not limited to other monoclonal antibodies not directed to CTLA4 (e.g., AVASTIN (bevacizumab), MYELOTARG (gemtuzumab), BEXXAR (tositumomab), RITUXAN (rituximab), HERCEPTIN (trastuzumab)), including antibodies that enhance the immune response (e.g., anti-CD40 agonistic antibodies), or protein ligands having similar effects; agents that activate antigen presenting cells (dendritic cells, macrophages, B cells, monocytes), including type 1 interferons (e.g., interferon alpha and beta); interferon gamma; BCG; agents that provide tumor antigens in any and all forms, including protein antigens, peptide antigens, whole cell lysates and derivatives thereof; genetically encoded antigens (e.g., adenovirus encoded antigens); cellular components of the immune system that have been altered either in vivo or ex vivo to enhance their immune properties (e.g., autologous dendritic cells, lymphocytes, heat shock proteins, etc.); chemotherapeutic agents such as, but not limited to, cyclophosphamide, methotrexate, etoposide, adriamycin, taxanes, fluorouracil, cytosine arabinoside (AraC), and platinum-containing agents, among numerous others.

I. Anti-CTLA4 Antibodies

As stated previously elsewhere herein, the preferred anti-CTLA4 antibody is a human antibody that specifically binds to human CTLA4. Exemplary human anti-CTLA4 antibodies are described in detail in International Application No. PCT/US99/30895, published on Jun. 29, 2000 as WO 00/37504, European Patent Appl. No. EP 1262193 A1, published Apr. 12, 2002, and U.S. patent application Ser. No. 09/472,087, now issued as U.S. Pat. No. 6,682,736, to Hanson et al., as well as U.S. patent application Ser. No. 09/948,939, published as US2002/0086014, the entire disclosure of which is hereby incorporated by reference. Such antibodies include, but are not limited to, 3.1.1, 4.1.1, 4.8.1, 4.10.2, 4.13.1, 4.14.3, 6.1.1, ticilimumab, 11.6.1, 11.7.1, 12.3.1.1, and 12.9.1.1, as well as ipilimumab. Human antibodies provide a substantial advantage in the treatment methods of the present invention, as they are expected to minimize the immunogenic and allergic responses that are associated with use of non-human antibodies in human patients.

Characteristics of useful human anti-CTLA4 antibodies of the invention are extensively discussed in WO 00/37504, EP 1262193, and U.S. Pat. No. 6,682,736 as well as U.S. Patent Application Publication Nos. US2002/0086014 and US2003/0086930, and the amino and nucleic acid sequences set forth therein are incorporated by reference herein in their entirety. Briefly, the antibodies of the invention include antibodies having amino acid sequences of the heavy and light chains of an antibody such as, but not limited to, antibody 3.1.1, 4.1.1, 4.8.1, 4.10.2, 4.13.1, 4.14.3, 6.1.1, ticilimumab, 11.6.1, 11.7.1, 12.3.1.1, 12.9.1.1, and ipilimumab. The invention also relates to antibodies having the amino acid sequences of the CDRs of the heavy and light chains of these antibodies, as well as those having changes in the CDR regions, as described in the above-cited applications and patent. The invention also concerns antibodies having the variable regions of the heavy and light chains of those antibodies. In another embodiment, the antibody is selected from an antibody having the full length, variable region, or CDR, amino acid sequences of the heavy and light chains of antibodies 3.1.1, 4.1.1, 4.8.1, 4.10.2, 4.13.1, 4.14.3, 6.1.1, ticilimumab, 11.6.1, 11.7.1, 12.3.1.1, and 12.9.1.1, and ipilimumab.

In one embodiment, the invention comprises an antibody-therapeutic agent combination comprising a human anti-CTLA4 antibody disclosed in U.S. patent application Ser. No. 09/948,939, published as U.S. Patent Application Publication No. 2002/0086014 and No. 2003/0086930, and references cited therein, including, but not limited to, MAb 10D1 (ipilimumab, Medarex, Princeton, N.J.). Even more preferably, the anti-CTLA4 antibody is ipilimumab.

In another embodiment, the amino acid sequence of the V_{H} comprises the amino acid sequences set forth in SEQ ID NOs:3, 15 and 27. In yet another embodiment, the V_{L} comprises the amino acid sequences set forth in SEQ ID NOs:9, 21 and 33. More preferably, the V_{H} and V_{L} comprise the amino acid sequences set forth in SEQ ID NO:3 (V_{H} 4.1.1) and SEQ ID NO:9 (V_{L} 4.1.1), respectively; the amino acid sequences set forth in SEQ ID NO:15 (V_{H} 4.13.1) and SEQ ID NO:21 (V_{L} 4.13.1), respectively; and the amino acid sequences set forth in SEQ ID NO:27 (V_{H} 11.2.1) and SEQ ID NO:33 (V_{L} 11.2.1), respectively.

In yet another embodiment, the amino acid sequence of the heavy chain comprises the amino acid sequence encoded by a nucleic acid comprising the nucleic acid sequences set forth in SEQ ID NOs:1, 13, and 25. In yet another embodiment, the light chain comprises the amino acid sequence encoded by a nucleic acid comprising the nucleic acid sequences set forth in SEQ ID NOs:7, 19 and 31. More preferably, the heavy and light chains comprise the amino acid sequences encoded by nucleic acids comprising the nucleic acid sequences set forth in SEQ ID NO:1 (heavy chain 4.1.1) and SEQ ID NO:7 (light chain 4.1.1), respectively; the nucleic acid sequences set forth in SEQ ID NO:13 (heavy chain 4.13.1) and SEQ ID NO:19 (light chain 4.13.1), respectively; and the nucleic acid sequences set forth in SEQ ID NO:25 (heavy chain 11.2.1) and SEQ ID NO:31 (light chain 11.2.1), respectively.

Furthermore, the antibody can comprise a heavy chain amino acid sequence comprising human CDR amino acid sequences derived from the V_{H} 3-30 or 3-33 gene, or conservative substitutions or somatic mutations therein. The antibody can also comprise CDR regions in its light chain derived from the A27 or 012 gene, i.e., fewer than five, or fewer than ten such mutations. The antibody can also comprise framework regions from those genes, including those that differ by fewer than five, or fewer than ten amino acids. Also included are antibodies with framework regions described herein that have been mutated to reflect the original germ-line sequence.

In other embodiments of the invention, the antibody inhibits binding between CTLA4 and B7-1, B7-2, or both. Preferably, the antibody can inhibit binding with B7-1 with an IC₅₀ of about 100 nM or lower, more preferably, about 10 nM or lower, for example about 5 nM or lower, yet more preferably, about 2 nM or lower, or even more preferably, for example, about 1 nM or lower. Likewise, the antibody can inhibit binding with B7-2 with an IC₅₀ of about 100 nM or lower, more preferably, 10 nM or lower, for example, even more preferably, about 5 nM or lower, yet more preferably, about 2 nM or lower, or even more preferably, about 1 nM or lower.

Further, in another embodiment, the anti-CTLA4 antibody has a binding affinity for CTLA4 of about 10⁻⁸, or greater affinity, more preferably, about 10⁻⁹ or greater affinity, more preferably, about 10⁻¹⁰ or greater affinity, and even more preferably, about 10⁻¹¹ or greater affinity.

The anti-CTLA4 antibody can compete for binding with an antibody having heavy and light chain amino acid sequences of an antibody selected from the group consisting of 4.1.1, 6.1.1, ticilimumab, 4.13.1 and 4.14.3. Further, the anti-CTLA4 antibody can compete for binding with an ipilimumab antibody.

In another embodiment, the antibody preferably cross-competes with an antibody having a heavy and light chain sequence, a variable heavy and a variable light chain sequence, and/or the heavy and light CDR sequences of antibody 4.1.1, 4.13.1, 4.14.3, 6.1.1. or ticilimumab. For example, the antibody can bind to the epitope to which an antibody that has heavy and light chain amino acid sequences, variable sequences and/or CDR sequences, of an antibody selected from the group consisting of 4.1.1, 4.13.1, 4.14.3, 6.1.1, or ticilimumab binds. In another embodiment, the antibody cross-competes with an antibody having heavy and light chain sequences, or antigen-binding sequences, of MDX-D010.

In another embodiment, the invention is practiced using an anti-CTLA4 antibody that comprises a heavy chain comprising the amino acid sequences of CDR-1, CDR-2, and CDR-3, and a light chain comprising the amino acid sequences of CDR-1, CDR-2, and CDR-3, of an antibody selected from the group consisting of 3.1.1, 4.1.1, 4.8.1, 4.10.2, 4.13.1, 4.14.3, 6.1.1, ticilimumab, 11.6.1, 11.7.1, 12.3.1.1, and 12.9.1.1, or sequences having changes from said CDR sequences selected from the group consisting of conservative changes, wherein the conservative changes are selected from the group consisting of replacement of nonpolar residues by other nonpolar residues, replacement of polar charged residues other polar uncharged residues, replacement of polar charged residues by other polar charged residues, and substitution of structurally similar residues; non-conservative substitutions, wherein the non-conservative substitutions are selected from the group consisting of substitution of polar charged residue for polar uncharged residues and substitution of nonpolar residues for polar residues, additions and deletions.

In a further embodiment of the invention, the antibody contains fewer than 10, 7, 5, or 3 amino acid changes from the germline sequence in the framework or CDR regions. In another embodiment, the antibody contains fewer than 5 amino acid changes in the framework regions and fewer than 10 changes in the CDR regions. In one preferred embodiment, the antibody contains fewer than 3 amino acid changes in the framework regions and fewer than 7 changes in the CDR regions. In a preferred embodiment, the changes in the framework regions are conservative and those in the CDR regions are somatic mutations.

In another embodiment, the antibody has at least 80%, more preferably, at least 85%, even more preferably, at least 90%, yet more preferably, at least 95%, more preferably, at least 99%, sequence identity over the heavy and light chain CDR-1, CDR-2 and CDR-3 sequences with the CDR sequences of antibody 3.1.1, 4.1.1, 4.8.1, 4.10.2, 4.13.1, 4.14.3, 6.1.1, ticilimumab, 11.6.1, 11.7.1, 12.3.1.1, and 12.9.1.1. Even more preferably, the antibody shares 100% sequence identity over the heavy and light chain CDR-1, CDR-2 and CDR-3 with the sequence of antibody 3.1.1, 4.1.1, 4.8.1, 4.10.2, 4.13.1, 4.14.3, 6.1.1, ticilimumab, 11.6.1, 11.7.1, 12.3.1.1, and 12.9.1.1.

In yet another embodiment, the antibody has at least 80%, more preferably, at least 85%, even more preferably, at least 90%, yet more preferably, at least 95%, more preferably, at least 99%, sequence identity over the heavy and light chain variable region sequences with the variable region sequences of antibody 3.1.1, 4.1.1, 4.8.1, 4.10.2, 4.13.1, 4.14.3, 6.1.1, ticilimumab, 11.6.1, 11.7.1, 12.3.1.1, and 12.9.1.1. Even more preferably, the antibody shares 100% sequence identity over the heavy and light chain variable region sequences with the sequences of antibody 3.1.1, 4.1.1, 4.8.1, 4.10.2, 4.13.1, 4.14.3, 6.1.1, ticilimumab, 11.6.1, 11.7.1, 12.3.1.1, and 12.9.1.1.

While the anti-CTLA4 antibodies discussed previously herein may be preferred, the skilled artisan, based upon the disclosure provided herein, would appreciate that the invention encompasses a wide variety of anti-CTLA4 antibodies and is not limited to these particular antibodies. More particularly, while human antibodies are preferred, the invention is in no way limited to human antibodies; rather, the invention encompasses useful antibodies regardless of species origin, and includes, among others, chimeric humanized and/or primatized antibodies. Also, although the antibodies exemplified herein were obtained using a transgenic mammal, e.g., a mouse comprising a human immune repertoire, the skilled artisan, based upon the disclosure provided herein, would understand that the present invention is not limited to an antibody produced by this or by any other particular method. Instead, the invention includes an anti-CTLA4 antibody produced by any method, including, but not limited to, a method known in the art (e.g., screening phage display libraries, and the like) or to be developed in the future for producing an anti-CTLA4 antibody of the invention. Based upon the extensive disclosure provided herein and in, e.g., U.S. Pat. No. 6,682,736, to Hanson et al., and U.S. Pat. App. Pub. No. 2002/0088014, one skilled in the art can readily produce and identify an antibody useful for treatment of breast cancer in combination with a therapeutic agent using the novel methods disclosed herein.

The present invention encompasses human antibodies produced using a transgenic non-human mammal, i.e., XenoMouse™ (Abgenix, Inc., Fremont, Calif.) as disclosed in the U.S. Pat. No. 6,682,736, to Hanson et al.

Another transgenic mouse system for production of “human” antibodies is referred to as “HuMAb-Mouse™” (Medarex, Princeton, N.J.), which contain human immunoglobulin gene miniloci that encodes unrearranged human heavy (mu and gamma) and kappa light chain immunoglobulin sequences, together with targeted mutations that inactivate the endogenous mu and kappa chain loci (Lonberg et al. Nature 368:856-859 (1994), and U.S. Pat. No. 5,770,429).

However, the invention uses human anti-CTLA4 antibodies produced using any transgenic mammal such as, but not limited to, the Kirin TC Mouse™ (Kirin Beer Kabushiki Kaisha, Tokyo, Japan) as described in, e.g., Tomizuka et al., Proc Natl Acad Sci USA 97:722 (2000); Kuroiwa et al., Nature Biotechnol 18:1086 (2000); U.S. Patent Application Publication No. 2004/0120948, to Mikayama et al.; and the HuMAb-Mouse™ (Medarex, Princeton, N.J.) and XenoMouse™ (Abgenix, Inc., Fremont, Calif.), supra. Thus, the invention encompasses using an anti-CTLA4 antibody produced using any transgenic or other non-human animal.

Moreover, while the preferred method of producing a human anti-CTLA-4 antibody comprises generation of the antibodies using a non-human transgenic mammal comprising a human immune repertoire, the present invention is in no way limited to this approach. Rather, as would be appreciated by one skilled in the art once armed with the disclosure provided herein, the invention encompasses using any method for production of a human, or any other antibody specific for CTLA4 produced according to any method known in the art or to be developed in the future for production of antibodies that specifically bind an antigen of interest

Human antibodies can be developed by methods that include, but are not limited to, use of phage display antibody libraries. Using these techniques, antibodies can be generated to CTLA4 expressing cells, CTLA4 itself, forms of CTLA4, epitopes or peptides thereof, and expression libraries thereto (see e.g. U.S. Pat. No. 5,703,057), which can thereafter be screened for the activities described above.

In another embodiment, the antibodies employed in methods of the invention are not fully human, but “humanized”. In particular, murine antibodies or antibodies from other species can be “humanized” or “primatized” using techniques well known in the art. See, e.g., Winter and Harris Immunol. Today 14:43-46 (1993), Wright et al. Crit. Reviews in Immunol. 12:125-168 (1992), and U.S. Pat. No. 4,816,567, to Cabilly et al, and Mage and Lamoyi in Monoclonal Antibody Production Techniques and Applications pp. 79-97, Marcel Dekker, Inc., New York, N.Y. (1987).

As will be appreciated based upon the disclosure provided herein, antibodies for use in the invention can be obtained from a transgenic non-human mammal, and hybridomas derived therefrom, but can also be expressed in cell lines other than hybridomas.

Mammalian cell lines available as hosts for expression are well known in the art and include many immortalized cell lines available from the American Type Culture Collection (ATCC), including but not limited to Chinese hamster ovary (CHO) cells, NSO (also referred to as NS0), HeLa cells, baby hamster kidney (BHK) cells, monkey kidney cells (COS), and human hepatocellular carcinoma cells (e.g., Hep G2). Non-mammalian prokaryotic and eukaryotic cells can also be employed, including bacterial, yeast, insect, and plant cells.

Various expression systems can be used as well known in the art, such as, but not limited to, those described in e.g., Sambrook and Russell, Molecular Cloning, A Laboratory Approach, Cold Spring Harbor Press, Cold Spring Harbor, N.Y. (2001), and Ausubel et al., Current Protocols in Molecular Biology, John Wiley & Sons, NY (2002). These expression systems include dihydrofolate reductase (DHFR)-based systems, among many others. The glutamine synthetase system of expression is discussed in whole or part in connection with European Patents Nos. EP 216 846, EP 256 055, and EP 323 997 and European Patent Application 89303964. In one embodiment, the antibody used is made in NS0 cells using a glutamine synthetase system (GS-NS0). In another embodiment, the antibody is made in CHO cells using a DHFR system. Both systems are well-known in the art and are described in, among others, Barnes et al. Biotech & Bioengineering 73:261-270 (2001), and references cited therein.

Site directed mutagenesis of the antibody CH2 domain to eliminate glycosylation may be preferred in order to prevent changes in either the immunogenicity, pharmacokinetic, and/or effector functions resulting from non-human glycosylation. Further, the antibody can be deglycosylated by enzymatic (see, e.g., Thotakura et al. Meth. Enzymol. 138:350 (1987)) and/or chemical methods (see, e.g., Hakimuddin et al., Arch. Biochem. Biophys. 259:52 (1987)).

Further, the invention encompasses using an anti-CTLA4 antibody comprising an altered glycosylation pattern. The skilled artisan would appreciate, based upon the disclosure provided herein, that an anti-CTLA4 antibody can be modified to comprise additional, fewer, or different glycosylations sites compared with the naturally-occurring antibody. Such modifications are described in, e.g., U.S. Patent Application Publication Nos. 2003/0207336, and 2003/0157108, and International Patent Publication Nos. WO 01/81405 and 00/24893.

Additionally, the invention comprises using an anti-CTLA4 antibody regardless of the glycoform, if any, present on the antibody. Moreover, methods for extensively remodeling the glycoform present on a glycoprotein are well-known in the art and include, e.g., those described in International Patent Publication Nos. WO 03/031464, WO 98/58964, and WO 99/22764, and US Patent Application Publication Nos. 2004/0063911, 2004/0132640, 2004/0142856, 2004/0072290, and U.S. Pat. No. 6,602,684 to Umaña et al.

Further, the invention encompasses using an anti-CTLA4 antibody with any art-known covalent and non-covalent modification, including, but not limited to, linking the polypeptide to one of a variety of nonproteinaceous polymers, e.g., polyethylene glycol, polypropylene glycol, or polyoxyalkylenes, in the manner set forth in, for example, U.S. Patent Application Publication Nos. 2003/0207346 and 2004/0132640, and U.S. Pat. Nos. 4,640,835; 4,496,689; 4,301,144; 4,670,417; 4,791,192; 4,179,337.

Additionally, the invention encompasses using an anti-CTLA4 antibody, or antigen-binding portion thereof, chimeric protein comprising, e.g., a human serum albumin polypeptide, or fragment thereof. Whether the chimeric protein is produced using recombinant methods by, e.g., cloning of a chimeric nucleic acid encoding the chimeric protein, or by chemical linkage of the two peptide portions, the skilled artisan would understand once armed with the teachings provided herein that such chimeric proteins are well-known in the art and can confer desirable biological properties such as, but not limited to, increased stability and serum half-life to the antibody of the invention and such molecules are therefore included herein.

Antibodies that are generated for use in the invention need not initially possess a particular desired isotype. Rather, the antibody as generated can possess any isotype and can be isotype switched thereafter using conventional techniques. These include direct recombinant techniques (see, e.g., U.S. Pat. No. 4,816,397), and cell-cell fusion techniques (see e.g., U.S. Pat. No. 5,916,771).

The effector function of the antibodies of the invention may be changed by isotype switching to an IgG1, IgG2, IgG3, IgG4, IgD, IgA, IgE, or IgM for various therapeutic uses. Furthermore, dependence on complement for cell killing can be avoided through the use of bispecifics, immunotoxins, or radiolabels, for example.

Therefore, while the preferred antibodies used in the invention are exemplified by antibodies having the amino acid sequences of 3.1.1, 4.1.1, 4.8.1, 4.10.2, 4.13.1, 4.14.3, 6.1.1, ticilimumab, 11.6.1, 11.7.1, 12.3.1.1, 12.9.1.1, and ipilimumab, or, e.g., the sequences of the V regions or CDRs thereof, the present invention is not limited in any way to using these, or any other, particular antibodies. The invention encompasses combining administration of any anti-CTLA4 antibody of the invention with an indolinone RTKI. Preferably, the antibody is 4.1.1, 4.13.1, ticilimumab, and/or ipilimumab. However, any anti-CTLA4 antibody, or antigen-binding portion thereof, as described elsewhere herein, or as known in the art or developed in the future, can be used in a method of the invention. More particularly, humanized chimeric antibodies, anti-CTLA4 antibodies derived from any species (including single chain antibodies obtained from camelids as described in, e.g., U.S. Pat. Nos. 5,759,808 and 6,765,087, to Casterman and Hamers), as well as any human antibody, can be combined with a therapeutic agent to practice the novel methods disclosed herein.

The invention also encompasses such antibodies as disclosed in, inter alia, International Patent Publication Nos. WO 00/37504 (published Jun. 29, 2000); WO 01/14424 (published Mar. 1, 2001); WO 93/00431 (published Jan. 7, 1993); and WO 00/32231 (published Jun. 8, 2000), among many others.

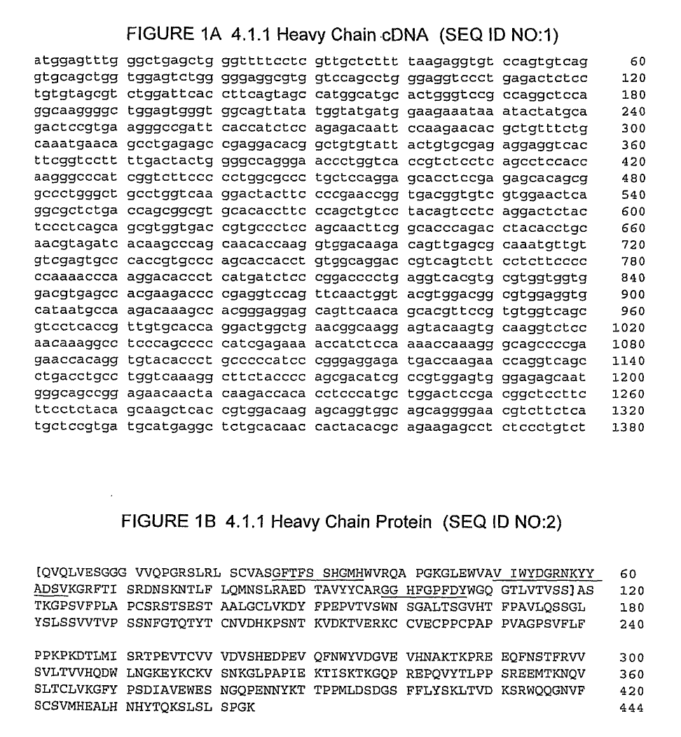

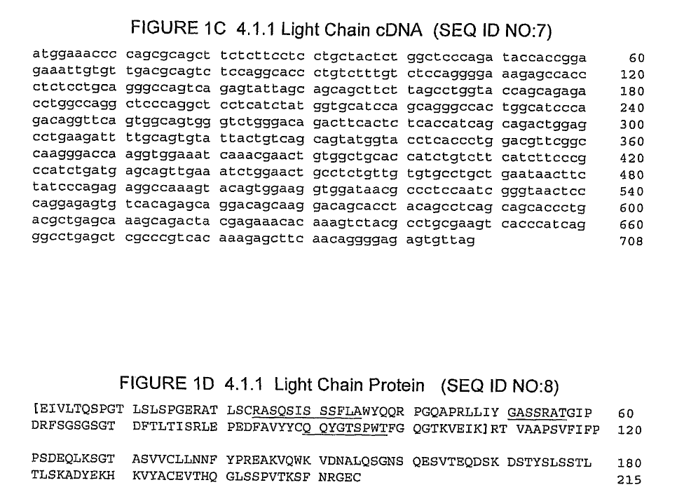

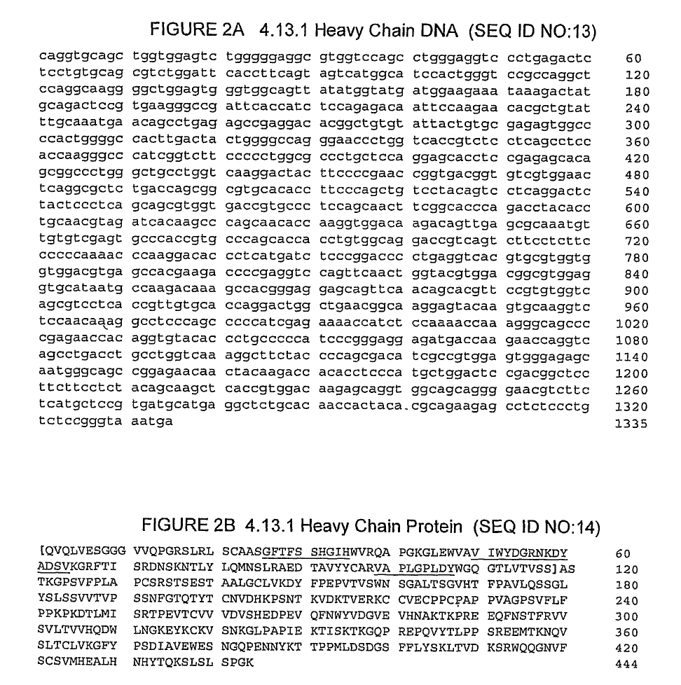

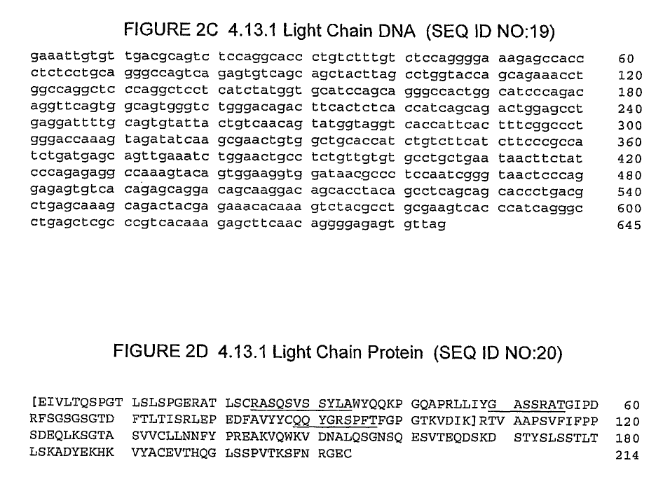

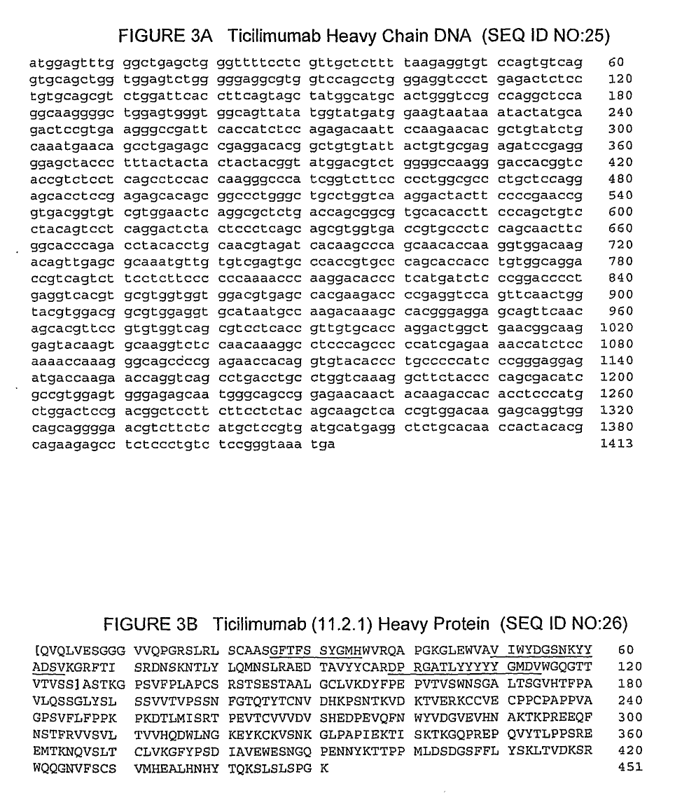

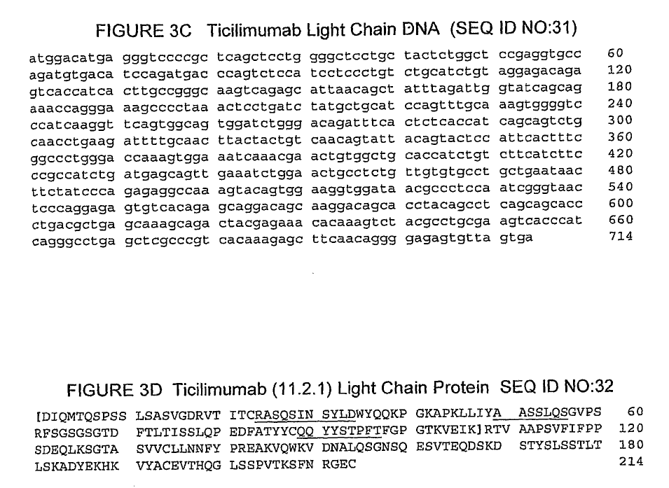

Although antibody 4.1.1, 4.13.1 and ticilimumab are IgG2 antibodies and the sequences of the variable regions of the antibodies are provided herein (FIGS. 1-3), and in the applications and patents referenced and incorporated herein, it is understood that the full-length sequences of these antibodies are encompassed herein, as well as the use of any antibody comprising the sequences set forth in SEQ ID NOs:1-36, and further comprising any constant region, regardless of isotype as more fully discussed elsewhere herein. Likewise, any antibody comprising the full-length sequence of ipilimumab, or any portion thereof, including a sequence encoding an antigen-binding portion of ipilimumab, can be administered in combination with an indolinone RTKI, e.g., sunitinib malate, thereby treating cancer.

Thus, the skilled artisan, once provided with the teachings provided herein, would readily appreciate that the anti-CTLA4 antibody-therapeutic agent combination of the invention can comprise a wide plethora of anti-CTLA4 antibodies. In one embodiment, the methods of the invention use ticilimumab. In another embodiment of the invention, the methods use anti-CTLA4 antibodies such as those described in, e.g., the following applications and patents: U.S. patent application Ser. No. 09/472,087, now issued as U.S. Pat. No. 6,682,736; Int. Appl. No. PCT/US99/30895 (published Jun. 29, 2000, as WO 00/37504); U.S. patent application Ser. No. 10/612,497 (published Nov. 18, 2004, as US 2004/0228858); U.S. patent application Ser. No. 10/776,649 (published Nov. 18, 2004, as US 2004/0228861); Int. Appl. No. Int. Appl. No. PCT/US00/23356 (published Mar. 1, 2001, as WO 01/14424) (e.g., antibody 10D1, also known as MDX-010, and ipilimumab, Medarex, Princeton, N.J.); Int. Appl. No. PCT/US99/28739 (published Jun. 8, 2000, as WO 00/32231); U.S. Pat. Nos. 5,811,097, 5,855,887, 6,051,227, and 6,207,156; U.S. Pat. No. 5,844,095, to Linsley et al.; Int. Appl. No. PCT/US92/05202 (published Jan. 7, 1993, as WO 93/00431); U.S. patent application Ser. No. 10/153,382 (published May 8, 2003, as US 2003/0086930); U.S. patent application Ser. No. 10/673,738 (published Feb. 24, 2005 as US 2005/0042223); U.S. patent application Ser. No. 11/085,368 (published Oct. 13, 2005, as US 2005/0226875); U.S. Pat. Appl. No. 60/624,856 (filed Nov. 4, 2004); U.S. Pat. Appl. No. 60/664,364 (filed Mar. 23, 2005); U.S. Pat. Appl. No. 60/664,653 (filed Mar. 23, 2005); U.S. Pat. Appl. No. 60/697,082 (filed Jul. 7, 2005); U.S. Pat. Appl. No. 60/711,707 (filed Aug. 26, 2005).

Further, one skilled in the art, based upon the disclosure provided herein, would understand that the invention is not limited to administration of only a single antibody; rather, the invention encompasses administering at least one anti-CTLA4 antibody, e.g., 4.1.1, 4.13.1, ticilimumab, and ipilimumab, in combination with a therapeutic agent. Moreover, the invention encompasses administering any combination of any known anti-CTLA4 antibody, including, but not limited to, administering a therapeutic agent in combination with, e.g., 4.1.1, 4.13.1, ticilimumab and ipilimumab. Thus, any combination of anti-CTLA4 antibodies can be combined with at least one therapeutic agent and the present invention encompasses any such combination and permutation thereof.

II. Antibody-Indolinone Combination Therapy

A. Indolinone RTKI

Exemplary indolinone RTKIs that can be used in the combination and method of the invention are described in, inter alia, International Publication Nos. WO 2003/016305 published Feb. 27, 2003 (US 2003/0069298 published Apr. 10, 2003), WO 2005/033098 published Apr. 14, 2005 (US 2005/0118255 published Jun. 2, 2005) and WO 2004/024127 published Mar. 25, 2004 (US 2004/0229930 published Nov. 18, 2004), and U.S. Pat. Nos. 6,573,293 and 6,653,308, the disclosures of which are incorporated herein by reference in their entireties. Thus, as would be understood by the skilled artisan armed with the teachings disclosed herein, although, in one embodiment, compound 1 is exemplified herein as a preferred indolinone, the present invention is not limited to these, or any other particular indolinone RTKI.

In one embodiment, the indolinone used in the invention has detectable activity in inhibiting at least one receptor tyrosine kinase, including, but not limited to, KIT, FLT3, VEGFR, and PDGFR. Even more preferably, the indolinone detectably inhibits KIT, FLT3, VEGFR, and PDGFR. Such indolinone RTKI used in combination with an anti-CTLA4 antibody comprises compound 1 and any metabolites thereof (e.g., compound 2), or compound 3. Preferably, the indolinone RTKI is compound 1 and compound 2. Even more preferably, the indolinone RTKI is compound 1. Any pharmaceutically acceptable salt of the compounds is included in the antibody-indolinone RTKI of the invention. Preferably, the salt of compound 1 is a malic acid salt, more preferably an L-malate salt. Preferably the salt of compound 3 is a maleic acid salt.

Exemplary indolinone RTKIs used in the present invention are described in International Publication No. WO 2003/016305 published Feb. 27, 2003, No. WO 2004/024127 published Mar. 25, 2004, No. WO 2004/045523 published Jun. 3, 2004, U.S. Pat. Nos. 6,573,293, and 6,653,308, and U.S. patent application Ser. No. 10/991,244 (published as US 2005/0182122 on Aug. 18, 2005), each of which is incorporated herein in its entirety for all purposes. Uses of these compounds are set forth in, among others, the references previously noted, as well as International Publication No. WO 2003/015608 published Feb. 27, 2003 (US 2003/0216410 published on Nov. 20, 2003) and No. WO 2004/045523 published Jun. 3, 2004 (US 2004/0152759 published Aug. 5, 2004), U.S. Patent Application Publication Nos. US 2005/0182122 (published Aug. 18, 2005), and in U.S. Provisional Application Nos. 60/719,119 (filed Sep. 20, 2005), 60/680,837 (filed May 12, 2005) and 60/753,797 (filed Dec. 23, 2005), all of which are incorporated by reference herein in their entireties.

In one embodiment, the indolinone RTKI is compound 1, referred to as N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide, and represented by formula 1 set forth previously herein (previously referred to as sunitinib malate, SU11248, SU011248 and SUTENT). The present invention encompasses an active metabolite of compound 1 (previously referred to as SU12662, which metabolite is referred to as N-[2-(ethylamino)ethyl]-5-[(Z)-(5-fluoro-2-oxo-1,2-dihydro-3H-indol-3-ylidene)methyl]-2,4-dimethyl-1H-pyrrole-3-carboxamide and is represented by formula 2 set forth previously herein. In another embodiment, the indolinone RTKI used is compound 3 referred to as 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, and represented by formula 3. As would be appreciated by the skilled artisan based upon the disclosure provided herein, any pharmaceutically acceptable salt of an indolinone RTKI can be used.

The indolinone compounds used in the present invention are pan inhibitors of protein kinases (PKs), and are therefore useful in the treatment of cancer because PKs are known to mediate and/or play a role in carcinogenesis. PKs whose catalytic activity is modulated by the indolinones used in the present invention include protein tyrosine kinases such as receptor tyrosine kinases (RTKs), cellular tyrosine kinases (CTKs), and serine-threonine kinases (STKs). RTK-mediated signal transduction is initiated by extracellular interaction with a specific growth factor (ligand), followed by receptor dimerization, transient stimulation of the intrinsic protein tyrosine kinase activity and phosphorylation. Binding sites are thereby created for intracellular signal transduction molecules and lead to the formation of complexes with a spectrum of cytoplasmic signaling molecules that facilitate the appropriate cellular response (e.g., cell division, metabolic effects on the extracellular microenvironment, etc.), as reviewed in, e.g., Schlessinger and Ullrich, 1992, Neuron 9:303-391.

It has been demonstrated that tyrosine phosphorylation sites on growth factor receptors function as high-affinity binding sites for SH2 (src homology) domains of signaling molecules. Fantl et al., 1992, Cell 69:413-423, Songyang et al., 1994, Mol. Cell. Biol. 14:2777-2785), Songyang et al., 1993, Cell 72:767-778, and Koch et al., 1991, Science 252:668-678. Several intracellular substrate proteins that associate with RTKs have been identified, and the specificity of the interactions between receptors and SH2 domains of their substrates is determined by the amino acid residues immediately surrounding the phosphorylated tyrosine residue (Songyang et al., 1993, Cell 72:767-778). Differences in the binding affinities between SH2 domains and the amino acid sequences surrounding the phosphotyrosine residues on particular receptors are consistent with the observed differences in their substrate phosphorylation profiles. Id. These observations suggest that the function of each RTK is determined not only by its pattern of expression and ligand availability but also by the array of downstream signal transduction pathways that are activated by a particular receptor. Thus, phosphorylation provides an important regulatory step which determines the selectivity of signaling pathways recruited by specific growth factor receptors, as well as differentiation factor receptors.

PK signal transduction results in, among other responses, cell proliferation, differentiation, growth and metabolism. Abnormal cell proliferation may result in a wide array of disorders and diseases, including the development of neoplasia such as carcinoma, sarcoma, glioblastoma and hemangioma, disorders such as leukemia, psoriasis, arteriosclerosis, arthritis and diabetic retinopathy and other disorders related to uncontrolled angiogenesis and/or vasculogenesis.

An understanding of the mechanism by which the indolinone compounds used in this invention is not required in order to practice the present invention. However, without wishing to be bound by any particular theory, the indolinone compounds used in the present invention may interact with the amino acids in the catalytic region of PKs. That is, PKs typically possess a bi-lobate structure wherein ATP appears to bind in the cleft between the two lobes in a region where the amino acids are conserved among PKs. Inhibitors of PKs, such as indolinone RTKI, are believed to bind by non-covalent interactions such as hydrogen bonding, van der Waals forces and/or ionic interactions in the same general region where the ATP otherwise binds to the PKs. More specifically, it is thought that the 2-indolinone component of the compounds used herein binds in the general space normally occupied by the adenine ring of ATP. Specificity of a particular molecule for a particular PK may then arise as the result of additional interactions between the various substituents on the 2-indolinone core and the amino acid domains specific to particular PKs. Thus, different indolinone substituents may contribute to preferential binding to particular PKs. The ability to select compounds active at different ATP (or other nucleotide) binding sites makes the indolinone compounds of the present invention particularly useful for targeting any protein with such a site. The compounds used in the present invention can therefore be used to mediate in vivo therapeutic effects through interaction with PKs.

The indolinone compounds used in the present invention combined with an anti-CTLA4 antibody, provide a powerful therapeutic approach to the treatment of many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia are also contemplated by this invention. Indications may include, but are not limited to brain cancers, bladder cancers, ovarian cancers, gastric cancers, pancreas cancers, colon cancers, kidney cancers, intestinal cancers, breast cancers, prostate cancers, blood cancers, lung cancers and bone cancers.

Additionally, without limitation, of the types of disorders related to inappropriate PK activity that the indolinone compounds used in the present invention when combined with an anti-CTLA4 antibody can be useful in preventing, treating and studying, are cell proliferative disorders, fibrotic disorders and metabolic disorders. Moreover, cell proliferative disorders, which may be prevented, treated or further studied by the present invention include cancer, blood vessel proliferative disorders and mesangial cell proliferative disorders.

B. Indolinone RTKI and Anti-CTLA4 Antibody Combination

The present invention relates to combination therapy comprising co-administering an indolinone PK inhibitor, preferably, indolinone RTKI, more preferably, the indolinone RTKI is compound 1, compound 2, or compound 3, and an anti-CTLA4 antibody, preferably, an antibody comprising an antigen-binding portion of antibody 4.1.1, 4.13.1, and ticilimumab, ipilimumab, among others. For purposes of this disclosure, reference to indolinone RTKIs, including references to compound 1, compound 2, or compound 3, include reference to their pharmaceutically acceptable salts, such as, but not limited to, the preferred salts described herein.

In one embodiment, a combination of an anti-CTLA4 antibody and an indolinone PK inhibitor is co-administered to a patient to treat cancer where the indolinone is compound 1.

Compound 1 is useful for treatment of, among other things, prostate cancer, ovarian cancer, thyroid cancer, melanoma, sarcoma, breast cancer, GIST (imatinib-resistant or not), NSCLC, pancreatic cancer, colorectal cancer, renal cell carcinoma, and the like. Combination of anti-CTLA4 antibody and indolinone RTKI is therefore useful for treatment of these cancers. More specifically, among many potential treatment options, indolinone RTKI and anti-CTLA4 combination therapy can be used to treat imatinib-resistant GIST, metastatic renal cell carcinoma, metastatic breast cancer, colorectal cancer, ovarian cancer, non-small cell lung cancer, metastatic breast cancer, neuroendocrine cancers (e.g., pancreatic islet cell cancer), among many others. While these cancers are preferred, the present invention relates to treatment of a wide variety of malignant cell proliferative disorders, including, but not limited to mesothelioma, hepatobilliary (hepatic and billiary duct), a primary or secondary CNS tumor, a primary or secondary brain tumor, lung cancer (NSCLC and SCLC), bone cancer, skin cancer, cancer of the head or neck, cutaneous or intraocular melanoma, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, chronic or acute myeloid leukemia, chronic or acute lymphocytic leukemia, lymphocytic lymphomas, cutaneous T cell lymphoma, cancer of the bladder, cancer of the kidney or ureter, renal cell carcinoma, carcinoma of the renal pelvis, neoplasms of the central nervous system (CNS), primary CNS lymphoma, non Hodgkin's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of the antibody-indolinone RTKI combination, the combination can be used to treat any abnormal cell growth, including a benign proliferative disease, such as, but not limited to, psoriasis, benign prostatic hypertrophy, and restenosis.

Furthermore, the invention encompasses use of an anti-CTLA4 antibody in combination with indolinone RTKI as a neoadjuvant, adjuvant, first line treatment, second-line and/or third-line therapy for cancer (e.g., adjuvant therapy for breast cancer, first line therapy for metastatic lung cancer, third line therapy for germ cell tumors, and the like). That is, in one embodiment, the antibody-indolinone RTKI combination can be co-administered as neoadjuvant therapy prior to, for instance, surgical resection of a tumor (e.g., prostate cancer). In another embodiment, the indolinone RTKI-antibody combination can be administered both as a neoadjuvant therapy (i.e., prior to surgery) and also following surgery as an adjuvant therapy. In yet another embodiment, the indolinone RTKI-antibody combination can be co-administered to treat metastatic renal cell carcinoma in a cytokine-refractory patient. Further, the combination can be used as a first-line treatment instead of another agent (e.g., interferon-alpha). Additional combinations and therapeutic regimens will be readily appreciated by one skilled in the art based upon the disclosure provided herein, including, but not limited to, co-administration of indolinone RTKI-anti-CTLA4 antibody combination to treat metastatic renal cell carcinoma as a second-line therapy in cytokine-refractory patients, as a second-line therapy in imatinib-resistant GIST patients, among many others. Combinations of these therapies, where indolinone RTKI-anti-CTLA4 combination is co-administered, are also encompassed in the present invention, such as, but not limited to, where the combination is used for neoadjuvant, adjuvant, first-line, and second-line therapy, or any combination thereof.

In one embodiment, the combination of the invention is administered in further combination with a standard of care therapy for one of the cancers described above. In another embodiment, the combination is administered to a patient who has failed standard of care therapy.

The skilled artisan would appreciate, once provided the teachings disclosed herein, that the invention encompasses indolinone RTKI therapy combined with immunotherapy using an anti-CTLA4 antibody with, or sequentially (preceding or following) with surgery, radiotherapy, or both, to treat cancer. That is, various treatments can be combined with sunitib-anti-CTLA4 combination therapy, as would be understood by one skilled in the art once armed with the teachings provided herein.

In another embodiment, an indolinone-RTKI, preferably, compound 1, compound 2 and compound 3, and an anti-CLTA-4 antibody combination is co-administered to enhance, prolong, or both, an immune response to a tumor. This is because there may be an interaction between the anti-tumor effect of indolinone RTKI and the anti-CTLA4 antibody of the invention that leads to more effective anti-tumor effect than either agent alone. Thus, without wishing to be bound by any particular theory, the combination of indolinone RTKI and anti-CTLA4 antibody can induce a more robust immunological response within the tumor than expected. Without wishing to be bound by any particular theory, the release of tumor antigen(s) mediated by the anti-tumor effect of the RTKI, e.g., indolinone RTKI, can increase the immunotherapeutic effect of an anti-CTLA4 which may be directed against such antigen(s). This is likely in that CTLA4 blockade using an antibody has been demonstrated to break tolerance (e.g., reverse or prevent anergy or tolerization to tumor antigens) to tumor antigens thereby rendering the tumor cells more susceptible to immune attack. Therefore, the combination of indolinone RTKI with an anti-CLTA-4 antibody can provide a potential synergistic effect thereby providing an important novel therapeutic treatment for cancer.

In one embodiment, the invention provides a compositions and methods of producing or increasing an anti-tumor response using an anti-CTLA4 antibody-indolinone RTKI combination, wherein the indolinone RTKI enhances an anti-tumor response by an amount of antibody which is otherwise sub-optimal for inducing the same level of anti-tumor response when used alone. In certain embodiments, when the indolinone RTKI is not used in conjunction with an antibody to elicit an anti-tumor response, administering indolinone RTKI alone does not produce or increase the anti-tumor response. In alternate embodiments, both the indolinone RTKI and the anti-CTLA4 antibody can elicit an anti-tumor response alone and/or when administered in combination.

In certain embodiments, the indolinone RTKI may enhance the effects of the anti-CTLA4 antibody in an additive manner. In a preferred embodiment, the indolinone RTKI enhances the effects of the anti-CTLA4 antibody in a synergistic manner. In another embodiment, the anti-CTLA4 antibody enhances the effect of an indolinone RTKI in an additive manner. Preferably, the effects are enhanced in a synergistic manner. Thus, in certain embodiments, the invention encompasses methods of disease treatment or prevention that provide better therapeutic profiles than administration of indolinone RTKI alone and/or anti-CTLA4 antibody alone.

Encompassed by the invention are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain embodiments, the methods of the invention permit treatment or prevention of diseases and disorders wherein treatment is improved by an enhanced anti-tumor response using lower and/or less frequent doses of anti-CTLA4 antibody and/or indolinone RTKIs to reduce the incidence of unwanted or adverse effects caused by the administration of anti-CTLA4 antibody and/or indolinone RTKIs alone, while maintaining or enhancing efficacy of treatment, preferably increasing patient compliance, improving therapy and/or reducing unwanted or adverse effects.

The methods and compositions of the invention are useful not only in untreated patients but are also useful in the treatment of patients partially or completely unresponsive to indolinone RTKIs administered alone or anti-CTLA4 antibody administered alone. In various embodiments, the invention provides methods and compositions useful for the treatment of diseases or disorders in patients that have been shown to be or may be refractory or non-responsive to therapies comprising the administration of either or both anti-CTLA4 antibody and/or indolinone RTKIs, and wherein treatment is improved by an enhanced immune response. In one embodiment, the method comprises combining an indolinone RTKI (preferably, compound 1) and an anti-CTLA4 antibody (preferably, antibody 4.1.1, antibody 4.13.1, ticilimumab, ipilimumab, or any combination thereof).

Indolinone RTKI can be administered according to standard dosing regimens well-known in the art such as, but not limited to, those described in U.S. patent application Ser. No. 10/991,244 (published as No. US2005/0182122 on Aug. 18, 2005). Briefly, in one embodiment of the invention, indolinone RTKI, more preferably, compound 1, is administered continuously once per day. In another embodiment, indolinone RTKI is administered continuously every day for about four weeks and no additional indolinone RTKI is administered. In one embodiment of the invention, the indolinone RTKI is administered continuously for a period greater than four weeks.

In one embodiment of the invention, indolinone RTKI is administered according to an intermittent dosing regimen comprising at least one cycle of an administration (or “treatment”) period followed by a “resting” period wherein the indolinone RTKI is not administered. In another embodiment, indolinone RTKI is administered for approximately four weeks followed by a resting period of about two weeks when the agent is not administered and then followed by at least one additional cycle of administration/resting. In another embodiment, indolinone RTKI is administered for approximately two weeks followed by a resting period of about one week where the agent is not administered followed by at least one additional administration/resting cycle. In a further embodiment, indolinone RTKI is administered for approximately three weeks followed by a resting period of about one week where the agent is not administered followed by at least one additional administration/resting cycle. The cycle of administration and resting period can then be repeated at least once, preferably, the administration/resting cycle is repeated twice. In another embodiment, the administration/resting cycle is adjusted such that any combination of the administration/resting cycles is administered. For instance, the indolinone RTKI is administered according to a 4/2 dosing regimen (i.e., four weeks administration followed by two week resting period) and a subsequent cycle of 3/1 is then administered. Thus, the invention encompasses any combination of intermittent dosing regimens as may be indicated for the patient. In one embodiment, administration of indolinone RTKI, whether by continuous or intermittent dosing regimen, is continued until progression of the disease, e.g., treatment can continue for at least two years. Preferred dosing regimens for compound 1 are described in detail in U.S. patent application Ser. No. 10/991,244, filed Nov. 17, 2004 (published as US 2005/0182122 on Aug. 18, 2005), the disclosure of which is incorporated herein by reference in its entirety.

In one embodiment, indolinone RTKI is administered once per day in an amount ranging from about 25 mg to 87.5 mg. Even more preferably, indolinone RTKI (e.g., compound 1) is administered once per day at about 37.5 mg to 50 mg. As used herein, reference to amounts of indolinone RTKIs, including compounds 1, 2 and 3, indicates the free base equivalent mass.

In one embodiment, the indolinone RTKI (e.g., compound 1, 2, and 3) and the antibody are co-administered in that indolinone is administered daily for four weeks, followed by two weeks rest, and the cycle is repeated, and wherein the antibody is administered after an appropriate rest period. Even more preferably, indolinone is administered orally at about 50 mg and the antibody is administered by i.v. infusion at a dose ranging from about 0.1 mg/kg to 50 mg/kg, more preferably, from about 0.3 mg/kg to 20 mg/kg, more preferably, from about 1 mg/kg to 15 mg/kg, even more preferably from about 3 mg/kg to 15 mg/kg, even more preferably, from about 6 mg/kg to 15 mg/kg.

In another embodiment, the antibody is administered at a dose of at least 0.3 mg/kg, preferably, at least 1 mg/kg, more preferably, at least 3 mg/kg, yet more preferably, at least 5 mg/kg, preferably, at least 6 mg/kg, even more preferably, at least 10 mg/kg, yet more preferably, at least 15 mg/kg, and even more preferably, at least 20 mg/kg.

In one embodiment of the invention, the antibody is administered at about 6 mg/kg every twenty-eight days. In another embodiment, the antibody is administered at about 6 mg/kg every three months. In one embodiment of the present invention, the antibody is administered at about 10 mg/kg every twenty-eight (28) days. In a further embodiment, the antibody is administered at about 10 mg/kg every three months. In another embodiment, the antibody is administered at about 15 mg/kg every twenty-eight days. In another embodiment, the antibody is administered at about 15 mg/kg every three months.

In one embodiment, the indolinone RTKI (e.g., compound 1, 2, and 3) and the antibody are co-administered in that indolinone is administered daily for four weeks, followed by two weeks rest, and the cycle is repeated, and the antibody is administered at day 1 of administration of the indolinone and then again every twenty eight days thereafter for at least one cycle of antibody administration where the antibody is administered at 10 mg/kg. In another embodiment, the indolinone RTKI is administered at an intermittent dosing regimen of 4/2 and the antibody is administered every three months at 10 mg/kg. In a further embodiment, the indolinone RTKI is administered at an intermittent dosing regimen of 4/2 and the antibody is administered every twenty-eight days at 15 mg/kg. In one embodiment of the invention, the indolinone RTKI is administered at an intermittent dosing regimen of 4/2 and the antibody is administered every three months at 15 mg/kg. In another embodiment of the present invention, indolinone RTKI (e.g., compound 1) is administered continuously at about 37.5 mg daily and the antibody is administered every three months at about 10 mg/kg. In yet another embodiment of the invention, indolinone RTKI (e.g., compound 1) is administered continuously at about 37.5 mg daily and the antibody is administered every three months at about 15 mg/kg. In yet a further embodiment of the present invention, indolinone RTKI (e.g., compound 1) is administered continuously at about 37.5 mg daily and the antibody is administered every three months at about 10 mg/kg and/or 15 mg/kg. That is, the dose of the antibody is varied and can comprise 10 or 15 mg/kg every three months as indicated.

Without wishing to be bound by any particular theory, indolinone RTKI may potentially cause detectable immune suppression in a patient in that temporary lymphopenia can occur upon administration of indolinone RTKI. Accordingly, in one embodiment of the present invention, the antibody can be administered approximately one to about one-hundred days following the last dose of indolinone RTKI. That is, the immune responsiveness of a patient (e.g., the level lymphopenia, if any, can be determined, among many other assays) can be assessed once a therapeutic treatment with an indolinone RTKI (e.g., compounds 1, 2, and 3) has been completed. The immune response, if decreased, may be assessed and an anti-CTLA4 antibody can be administered once the immune response has detectably increased compared to the level of the response during and/or immediately following indolinone RTKI administration. The level of the immune response can, but need not return to the level prior to administration of indolinone RTKI to the patient.

In one embodiment, a course of an indolinone RTKI, preferably, compound 1, is administered to a patient in need thereof. Upon completion of the course, a suitable resting period is allowed to pass before administration of an anti-CTLA4. A suitable resting period encompasses any time during which the level of immune response, as assessed by a method known in the art, detectably increases compared with the level of immune response by the patient during or immediately following the course of indolinone RTKI. Exemplary intermittent dosing regimens comprising treatment and resting periods are described in U.S. patent application Ser. No. 10/991,244 (published as US 2005/0182122 on Aug. 18, 2005), which is incorporated by reference in its entirety herein for all purposes.

While any suitable resting period can be used to allow the immune response to increase following a course of indolinone RTKI, the present invention does not require any resting period between administration of the indolinone RTKI and the antibody, thus the antibody and indolinone RTKI can be co-administered substantially contemporaneously. The timing of administration of indolinone RTKI with respect to administration of the antibody is well within the expertise of one skilled in the relevant art based upon the teachings provided herein.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present invention. Further, one skilled in the art would understand, once armed with the teachings provided herein, that a therapeutic benefit, such as, but not limited to, detectable decrease in tumor size and/or metastasis, decreased level of PSA in prostate cancer, and increased time to recurrence, among many other parameters, can be assessed by a wide variety of methods known in the art for assessing the efficacy of treatment of cancer, and these methods are encompassed herein, as well as methods to be developed in the future.

While the present invention is exemplified by methods relating to adjuvant, first-line and/or second-line therapy comprising administering a combination comprising co-administration of an indolinone RTKI, e.g., compound 1, and an anti-CTLA4 antibody, the skilled artisan, armed with the teachings provided herein, would understand that the invention is not limited to any particular therapy. Rather, methods comprising combined indolinone

RTKI and anti-CTLA4 antibody therapy encompass use of the combination along the entire disease and treatment continuum. More specifically, the novel methods disclosed herein can provide a therapeutic benefit before and after metastasis, as well as to patients that have become refractory to a chemotherapeutic agent, in that the antibody can enhance an immune response, including any response mediated by therapy as well as any response mediated by indolinone RTKI.

Thus, the present invention is not limited to use of the combinations of the invention solely for neoadjuvant therapy; instead, the invention includes the entire treatment spectrum, including, but not limited to, adjuvant, first-line, and/or second-line therapy for cancer. This is because the data disclosed herein suggest that immunotherapy comprising an anti-CLTA-4 antibody can provide a therapeutic benefit either alone or combined with at least one additional agent, at any point during treatment. That is, the efficacy of a method that mediates release of tumor-specific antigens, such as cytotoxic therapies (e.g., radiation, chemotherapeutics, inhibition of PKs, and the like), where such antigens are exposed to the immune system, can be enhanced by administration of an anti-CTLA4 antibody of the invention. Indeed, the data disclosed herein further suggest that a synergistic effect is mediated by combined administration of the antibody with RTKI therapy for treatment of cancer, more particularly, prostate, breast, CRC, melanoma, pancreatic, lung, GIST, RCC, among many cancers. Therefore, the present invention provides important novel therapeutics for treatment of cancer whereby the patient's immune system is enhanced to provide an anti-tumor effect.

III. Additional Combination Therapy

Based upon the disclosure provided herein, including the immune-enhancing effect of administering an anti-CTLA4 antibody to a patient, and the combined additive or synergistic effect of co-administering such antibody in combination with an indolinone RTKI (preferably, compound 1), it would be appreciated by the skilled artisan that the invention encompasses numerous combination therapies wherein the antibody-indolinone RTKI is administered to the patient in combination with at least one other therapeutic agent thereby providing a therapeutic benefit. Although many such combinations will be readily apparent to one skilled in the art once armed with the teachings provided herein, several combinations are now discussed. However, the present invention is in no way limited to these combinations, which are set forth herein merely for illustrative purposes.

Co-administration of the antibody-indolinone with an additional therapeutic agent (combination therapy) encompasses co-administering both the anti-CTLA4 antibody, indolinone, and one or more additional therapeutic agents, and also encompasses co-administering two or more separate pharmaceutical compositions, one comprising the anti-CTLA4 antibody and the other(s) comprising the indolinone, and other(s) comprising at least one additional therapeutic agent. Further, although co-administration or combination (conjoint) therapy generally mean that the antibody, indolinone, and additional therapeutic agents are administered at the same time as one another, it also encompasses simultaneous, sequential or separate dosing of the individual components of the treatment. Additionally, where an antibody is administered intravenously and the anti-cancer agent is administered orally (e.g., indolinone RTKI, and the like), or by subcutaneous or intramuscular injection, it is understood that the combination is preferably administered as two, three, or more separate pharmaceutical compositions.

When a mammal is subjected to additional chemotherapy, chemotherapeutic agents well-known in the art can be used in combination with an anti-CTLA4. Additionally, growth factor inhibitors, biological response modifiers, alkylating agents, intercalating antibiotics, vinca alkaloids, immunomodulators, taxanes, selective estrogen receptor modulators (SERMs), such as, but not limited to, lasofoxifene, angiogenesis inhibitors, among many therapeutic agents, some of which are described below, can be used.

Angiogenesis Inhibitors

Use of an angiogenesis inhibitor in combination with an anti-CTLA4 antibody has been discussed previously elsewhere herein. Moreover, an angiogenesis inhibitor includes, but is not limited to, bevacizumab (AVASTIN; Genentech), a humanized antibody to VEGF. It can be used in combination with 5FU, and is indicated as a first-line treatment of patients with metastatic carcinoma of the colon or rectum. Agents that directly target angiogenic factors or their receptors offer the prospect for greater activity in receptor-competent hematologic malignancies by interrupting autocrine receptor signaling. Bevacizumab produces sustained neutralization of circulating VEGF and may be useful for treatment of myelodysplastic syndrome (MDS), lymphoma, acute myeloid leukemia (AML), and solid tumors. In addition to indolinone, other RTKI small molecule inhibitors of angiogenic receptor signaling are encompassed in the invention. The first receptor antagonist to enter clinical testing in hematologic malignancies is SU5416 (Sugen), which impairs ligand-induced autophosphorylation of the VEGFR-1 and VEGFR-2 receptors and c-Kit. SU5416 inhibits VEGF-induced clonogenic response in leukemia cell lines and promotes apoptosis in myeloblasts from AML patients. Other RTKIs, including PTK787/ZK222584 (Novartis), and AG-13736 (Agouron/Pfizer), are being assessed to treat AML and other receptor-competent hematologic malignancies. The invention also includes treatment of cancer, e.g., renal carcinoma, gastrointestinal stromal tumors, and the like, using a combination of an anti-CTLA4 antibody and an indolinone RTKI, e.g., compound 1, compound 2, and compound 3, and at least one additional angiogenesis inhibitor, e.g., AG-13736, AG-26,798, and the like, as well as other angiogenesis inhibitors that are well-known in the art or developed in the future.

Thus, anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with the antibody-indolinone RTKI combination of the invention. Examples of useful COX-II inhibitors include CELEBREX™ (celecoxib), valdecoxib, rofecoxib, parecoxib, deracoxib, SD-8381, ABT-963, etoricoxib, lumiracoxib, BMS-347070, NS-398, RS 57067, meloxicam. Examples of useful matrix metalloproteinase inhibitors are described in International Patent Publication Nos. WO 96/33172; WO 96/27583; WO 98/07697, WO 98/03516, WO 98/34918, WO 98/34915, WO 98/33768, WO 98/30566, WO 90/05719, WO 99/52910, WO 99/52889, WO 99/29667, European Patent Application Nos. 780386 (published Jun. 25, 1997), 97304971.1 (filed Jul. 8, 1997), 99308617.2 (filed Oct. 29, 1999), 606046 (published Jul. 13, 1994), 931788 (published Jul. 28, 1999), 99302232.1 (filed Mar. 25, 1999), International Application PCT/IB98/01113 (filed Jul. 21, 1998), Great Britain patent application number 9912961.1 (filed Jun. 3, 1999), U.S. Provisional Patent Application No. 60/148,464 (filed Aug. 12, 1999), and U.S. Pat. Nos. 5,863,949, and 5,861,510.

Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Signal Transduction Inhibitor

The treatments described herein can also be used with signal transduction inhibitors other than indolinone RTKI (e.g., compound 1), such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN (Genentech, Inc., San Francisco, Calif.).

EGFR inhibitors are described in, for example in International Patent Publication Nos. WO 95/19970, WO 98/14451, WO 98/02434, and U.S. Pat. No. 5,747,498, and such substances can be used in the present invention as described herein. EGFR-inhibiting agents include, but are not limited to, the monoclonal antibodies C225 (ERBITUX), anti-EGFR 22Mab (ImClone Systems Inc., New York, N.Y.), and ABX-EGF (panitumumab, Abgenix Inc., Fremont, Calif.), the compounds ZD-1839 (AstraZeneca), BIBX-1382 (Boehringer Ingelheim), MDX-447 (Medarex,lnc., Annandale, N.J.), and OLX-103 (Merck & Co., Whitehouse Station, N.J.), VRCTC-310 (Ventech Research) and EGF fusion toxin (Seragen Inc., Hopkinton, Mass.). These and other EGFR-inhibiting agents can be used in the present invention.

Compounds directed at inhibition of epidermal growth factor receptor (EGFR) tyrosine kinase (TK) represent a relatively new class of antineoplastic drugs that are useful in the method of the present invention. Many human cancers express members of the EGFR family on the cell surface. When a ligand binds to EGFR, it sets off a cascade of cellular reactions that result in increased cell division and influence other aspects of cancer development and progression, including angiogenesis, metastatic spread, and inhibition of apoptosis. EGFR-TK inhibitors may selectively target one of the members of the EGFR family (EGFR (also known as HER1 or ErbB-1), HER2/neu (also known as ErbB-2), HER3 (also known as ErbB-3), or HER4 (also known as ErbB-4)), or may target two or more of them. EGFR-TK inhibitors suitable for use in the present invention include gefitinib (IRESSA), erlotinib (TARCEVA), CI-1033 (Pfizer), GW2016 (GlaxoSmithKline), EKB-569 (Wyeth), PKI-166 (Novartis), CP-724,714 (Pfizer), and BIBX-1382 (Boeringer-Ingelheim). Additional EGFR-TK inhibitors are described in U.S. patent application Ser. No. 09/883,752, filed Jun. 18, 2001.

VEGF inhibitors, in addition to indolinone RTKI, for example SU-5416 and SU-6668 (Sugen Inc., San Francisco, Calif.), can also be employed in combination with the antibody and indolinone RTKI combination. VEGF inhibitors are described for example in International Patent Application No. PCT/IB99/00797 (filed May 3, 1999), International Patent Publication Nos. WO 99/24440; WO 95/21613; WO 99/61422; WO 98/50356; WO 99/10349; WO 97/32856; WO 97/22596; WO 98/54093; WO 98/02438; WO 99/16755; WO 98/02437; U.S. Pat. Nos. 5,834,504; 5,883,113; 5,886,020; and 5,792,783. Other examples of some specific VEGF inhibitors useful in the present invention are IM862 (Cytran Inc., Kirkland, Wash.); IMC-1C11 Imclone antibody, anti-VEGF monoclonal antibody of Genentech, Inc., San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc., Woodlands, Tex.) and 2B-1 (Chiron), can furthermore be combined with the antibody-indolinone RTKI combination, for example those indicated in International Patent Publication Nos. WO 98/02434; WO 99/35146; WO 99/35132; WO 98/02437; WO 97/13760; WO 95/19970; U.S. Pat. Nos. 5,587,458, and 5,877,305. ErbB2 receptor inhibitors useful in the present invention are also described in EP1029853 (published Aug. 23, 2000) and in International Patent Publication No. WO 00/44728, (published Aug. 3, 2000). The erbB2 receptor inhibitor compounds and substance described in the aforementioned PCT applications, U.S. patents, and U.S. provisional applications, as well as other compounds and substances that inhibit the erbB2 receptor, can be used with the antibody in accordance with the present invention.

The treatments of the invention also be used with other agents useful in treating abnormal cell growth or cancer, including, but not limited to other agents capable of enhancing antitumor immune responses, such as additional, different, CTLA4 antibodies, and other agents also capable of blocking CTLA4; and anti-proliferative agents such as farnesyl protein transferase inhibitors (e.g., BMS 214662), and ανβ3 inhibitors, such as the ανβ3 antibody VITAXIN, ανβ5 inhibitors, p53 inhibitors, and the like.

Where the antibody of the invention is administered in combination with another immunomodulatory agent, the immunomodulatory agent can be selected for example from the group consisting of a dendritic cell activator such as CD40 ligand and anti-CD40 agonist antibodies, as well as enhancers of antigen presentation, enhancers of T-cell tropism, inhibitors of tumor-related immunosuppressive factors, such as TGF-β (transforming growth factor beta), and IL-10. Preferred anti-CD40 agonist antibodies encompass antibodies disclosed in International Patent Application No. PCT/US02/36107, filed Nov. 8, 2002 (published as WO 03/040170 on May 15, 2003), and U.S. patent application Ser. No. 10/292,088, filed Nov. 8, 2002 (published as U.S. Patent Publication No. US2003/021 1100 on Nov. 13, 2003), including, but not limited to, an antibody having the heavy and light chain amino acid sequence of antibody 3.1.1, 3.1.1.H-A78T, 3.1.1H-A78T-V88A-V97A, 3.1.1 L-L4M-L83V, 3.1.1H-A78T-V88A-V97A/3.1.1L-L4M-L83V, 7.1.2, 10.8.3, 15.1.1, 21.2.1, 21.4.1, 22.1.1, 22.1.1H-C109A, 23.5.1, 23.25.1, 23.28.1, 23.28.1H-D16E, 23.29.1, and 24.2.1.

IGF-1R Inhibitor

The present treatment regimens may also be combined with antibodies or other ligands that inhibit tumor growth by binding to IGF-1R (insulin-like growth factor 1 receptor). Specific anti-IGF-1R antibodies that can be used in the present invention include those described in International Patent Application No. PCT/US01/51113, filed Dec. 20, 2001 (published as WO 02/053596 on Jul. 11, 2002), and International Patent Application No. PCT/IB2004/002555, filed Aug. 3, 2004 (published as WO 2005/016967 on Feb. 24, 2005). Preferred anti-IGFR-1R antibodies encompass an antibody having the heavy and light chain amino acid sequence of, e.g., antibody 2.12.1, 2.13.2, 2.14.3, 3.1.1, 4.9.2 and 4.17.3.

Ligands that inhibit signaling via the IGF-1R also encompass small molecules, and other ligands including, inter alia, somavert (PEGVISOMANT), which is a growth hormone analog that inhibits IGF-1 signaling. PEGVISOMANT is conjugated with polyethylene glycol and can be used, among other things, to treat acromegaly. PEGVISOMANT can be co-administered with anti-CTLA4 antibody to treat cancer in that the combination can inhibit tumor growth. Thus, PEGVISOMANT, similarly with anti-IGF-1R antibodies, can be used to treat cancer as disclosed herein.

The present invention encompasses methods comprising combination of RTKI therapy (preferably, compound 1) with immunotherapy (anti-CTLA4) further combined with additional agents and therapies. That is, the skilled artisan, based upon the disclosure provided herein, would appreciate that indolinone RTKI therapy and anti-CTLA4 antibody combination therapy can be further combined with a wide plethora of therapeutic, surgical, radiation, and other therapeutics, to treat a patient. Therapeutic agents are numerous and have been described in, for instance, U.S. Patent Application Publication No. 2004/0005318, No. 2003/0086930, No. 2002/0086014, and International Publication No. WO 03/086459, all of which are incorporated by reference herein, among many others. Such therapeutic agents include, but are not limited to, topoisomerase I inhibitors; other antibodies (rituximab, trastuzumab, anti-IGF-1R, and the like); chemotherapeutic agents such as, but not limited to, imatinib (GLEEVEC, GLIVEC, or STI571; Novartis), sorafenib (BAY 43-9006; Bayer Pharmaceuticals Corp./Onyx Pharmaceuticals), selective estrogen receptor modulators (SERMs), taxanes, vinca alkaloids, temozolomide, angiogenesis inhibitors, EGFR inhibitors, VEGF inhibitors, ErbB2 receptor inhibitors, anti-proliferative agents (e.g., farnesyl protein transferase inhibitors, and ανβ3 inhibitors, ανβ3 inhibitors, p53 inhibitors, and the like), immunomodulators, cytokines, tumor vaccines; tumor-specific antigens; dendritic and stem cell therapies; alkylating agents, folate antagonists; pyrimidine antagonists; anthracycline antibiotics; platinum compounds; costimulatory molecules (e.g., CD4, CD25, PD-1, B7-H3, 4-1BB, OX40, ICOS, CD30, HLA-DR, MHCII, and LFA).

Radiotherapy

Radiation therapy can be co-administered with indolinone RTKI/anti-CTLA4 antibody combination therapy. Radiotherapy is administered in accordance to well-known radiotherapy methods for treatment of breast cancer. The dose and regimen for radiotherapy can be readily determined by one skilled in the art and is based on the stage of the disease, and other factors well-known in the art.

Palliative Agents

The present invention also encompasses the administration of other therapeutic agents in addition to anti-CTLA4 antibody and hormonal therapy agents. Such therapeutic agents include analgesics, cancer vaccines, anti-vascular agents, anti-proliferative agents, anti-emetic agents, and anti-diarrheal agents. Preferred anti-emetic agents include ondansetron hydrochloride, granisetron hydrochloride, and metoclopramide. Preferred anti-diarrheal agents include diphenoxylate and atropine (LOMOTIL), loperamide (IMMODIUM), and octreotide (SANDOSTATIN).

In another embodiment, the invention includes administering an agent with anti-diarrheal effect wherein the agent is indicated in the treatment of chronic inflammatory conditions of the gastrointestinal tract. Such agents include, among others, steroids with topical activity (e.g., budesonide [ENTOCORT]), and anti-tumor necrosis factor (TNF) drugs (e.g., infliximab [REMICADE], etanercept [ENBREL], and adalimumab [HUMIRA]).

Stem Cell-Based Therapy

The antibody-indolinone RTKI therapy combination disclosed herein can be combined with stem cell transplantation to provide a therapeutic benefit to a patient afflicted with cancer. Stem cell transplantation may be performed according to the methods known in the art. Some such methods are described in Appelbaum in Harrison's Principles of Internal Medicine, Chapter 14, Braunwald et al., Eds., 15^{th} ed., McGraw-Hill Professional (2001), which is hereby incorporated herein by reference, Thus, the methods of the present invention relate to the treatment of cancer in a mammal who has undergone stem cell transplantation, which methods comprise administering to the mammal an amount of a human anti-CTLA4 antibody in combination with indolinone RTKI therapy (preferably, compound 1), which antibody-indolinone therapy combination is effective in treating the cancer in further combination with stem cell transplantation.

Where the method comprises stem cell transplant, the first dose of the antibody-indolinone therapy agent combination can be administered after the immune system of the mammal has recovered from transplantation, for example, in the period of from one to 12 months post transplantation. In certain embodiments, the first dose is administered in the period of from one to three, or one to four months post transplantation. The patient may undergo stem cell transplantation and preparatory treatment(s).

The invention also relates to a method for the treatment of cancer in a mammal comprising the steps of (i) performing stem cell transplantation in the mammal, and (ii) administering an effective amount of a human anti-CTLA4 antibody in combination with an effective amount of indolinone RTKI. Preferably, the mammal is a human. Stem cell transplantation may be allogeneic or autologous stem cell transplantation. Further, cell transplantation encompasses adoptive transfer of lymphocytes, either from the same patient and/or from a HLA-matched donor.

Further, the methods of the invention can be combined with radiation therapy and stem cell transplant, and any combination of any of the treatments described herein, known in the art, or to be developed in the future.

As pointed out previously elsewhere herein, where an anti-CTLA4 antibody is combined with a standard cancer treatment, such as, inter alia, chemotherapeutic regimes, it may be possible to reduce the dose of chemotherapeutic reagent administered (Mokyr, M. et al. Cancer Research 58: 5301-5304 (1998)). This is because combined use of an anti-CTLA4 antibody and chemotherapy, such as RTKI using indolinone RTKI as disclosed herein for treatment of cancer, can mediate cell death that is a consequence of the cytotoxic action of most chemotherapeutic compounds, or otherwise provide a synergistic effect between the immune system and androgen suppression for, inter alia, prostate cancer (Grossman, Science 227:257-261 (1985); Olsen and Kovacs, Immunologic Research 23:281-288 (2001); Tanriverdi et al., J. Clin. Endocrinol. 176:293-304 (2003). Without wishing to be bound by any particular theory, tumor cell death likely results in increased levels of tumor-specific antigen in the antigen presentation pathway, and the anti-CTLA4 antibody mediates an increased immune response thereto. Other combination therapies that can result in synergy with anti-CTLA4 enhancement of the immune response through cell death release of tumor-specific antigens are radiation, surgery, chemotherapy, and administration of a wide plethora of anti-tumor agents well-known in the art and as exemplified herein, among many others. Each of these protocols, and others described elsewhere herein, creates a source of tumor-specific antigen in the host by tumor cell death which may feed tumor antigen into host antigen presentation pathways. Therefore, the combination therapies disclosed herein can provide an increased source of tumor-specific antigens thereby providing an increased immune response to the tumor which, in turn, provides a therapeutic benefit to the patient.

IV. Dosage Regimens

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of sensitivity in individuals.

An exemplary, non-limiting range for a therapeutically effective amount of an antibody administered according to the invention is at least about 0.1 mg/kg, at least about 0.3 mg/kg, at least about 1 mg/kg, at least about 5 mg/kg, at least about 6 mg/kg, at least about 10 mg/kg, at least about 15 mg/kg, or at least about 20 mg/kg. For example, a therapeutically effective amount of antibody can range from about 0.1-30 mg/kg, or for example about 0.3-25 mg/kg, or for example about 1-20 mg/kg, or for example about 3-20 mg/kg, or for example about 5-20 mg/kg, or for example about 10-20 mg/kg, or about 3-15 mg/kg, or about 5-15 mg/kg, or about 10-15 mg/kg.

Further, an exemplary dose escalation protocol can be used to determine the maximum tolerated dose (MTD), to assess dose limiting toxicity (DLT), if any, associated with administration of antibody-indolinone RTKI combination therapy, and the like, comprises administering increasing doses, such as, but not limited to about 0.1 mg/kg, 0.3 mg/kg, 1 mg/kg, 3 mg/kg, 6 mg/kg, 7 mg/kg, 10 mg/kg, 12 mg/kg, 15 mg/kg, or more than 15 mg/kg, or any combination thereof, more preferably, successive doses of 0.1 mg/kg, 0.3 mg/kg, 1 mg/kg, 3 mg/kg, 6 mg/kg, 10 mg/kg, 15 mg/kg or 20 mg/kg are administered and the patient is assessed for toxicity, if any, as well as for efficacy of treatment, among other parameters. Such studies to determine toxicity and efficacy of dose regimens are well-known in the art.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Determining appropriate dosages and regimens for administration of the antibody are well-known in the relevant art and would be understood to be encompassed by the skilled artisan once provided the teachings disclosed herein.

In one embodiment, the antibody is administered in an intravenous formulation as a sterile aqueous solution containing about 5 to 20 mg/ml of antibody, in an appropriate buffer system.

In one embodiment, part of the dose is administered by an intraveneous bolus and the rest by infusion of the antibody formulation. For example, an intravenous injection of the antibody may be given as a bolus, and the rest of a predetermined antibody dose may be administered by intravenous injection. A predetermined dose of the antibody may be administered, for example, over a period of about an hour and a half to about five hours.

The present invention relates to administering a combination of an anti-CTLA4 antibody and indolinone RTKI. The skilled artisan would appreciate that the combination can be administered simultaneously or the antibody and various agents can be administered at different times. For instance, in one embodiment, the antibody is administered as a single injection and the a therapeutic agent (e.g., indolinone RTKI) is administered once per day for about 28 days, concurrently with administration of the antibody. Even more preferably, indolinone RTKI, preferably, compound 1, is administered daily, per os, for 28 days of the first cycle and is not administered for two weeks thereafter. Even more preferably, the antibody is administered after any substantial immunosuppressive effect(s) of indolinone RTKI subside. Methods for assessing the immunosuppressive effect of indolinone RTKI, as well as resolution of the effects, are well known in the art. Additional cycles of antibody and indolinone RTKI can be provided as determined by art-recognized methods. However, the present invention is not limited to these or any particular dosage or administration regimens for administering indolinone RTKI in combination with an anti-CTLA4 antibody. Rather, the optimal dose, route and regimen for administration of the antibody and indolinone RTKI can be readily determined by one of ordinary skill in the relevant art using well-known methods.

For instance, a single dose or multiples doses of the antibody may be administered. Alternatively, at least one dose, or at least three, six or 12 doses may be administered. The doses may be administered, for example, every two weeks, monthly, every twenty days, every 25 days, every 28 days, every 30 days, every 40 days, every 50 days, every two months, every 70 days, every 80 days, every three months, every six months or yearly. In addition, indolinone RTKI can be administered daily, several times or once per day, weekly, every other week, every third week, every fourth week, monthly, every three months, every six months, once per year, or any other period that provides a therapeutic benefit to the patient as determined by the skilled practitioner.

In one embodiment, a single bolus injection comprising the anti-CTLA4 antibody is administered to a patient intravenously at a dose ranging from about 1 mg/kg to 20 mg/kg approximately every twenty-eight days. A dose of indolinone RTKI is administered on that first day, and approximately every day for about twenty-eight days thereafter. Preferably, the antibody and indolinone RTKI are co-administered on the same starting day of each dose cycle. Further, the invention encompasses administering indolinone RTKI at any point during administration of the antibody, or vice-a-versa, and the invention is not limited in any way with respect to the relative administration of the antibody and indolinone RTKI. Thus, indolinone RTKI can be administered either before, during and/or after administration of the antibody.

The antibody-indolinone RTKI combination can be administered as a neoadjuvant therapy prior to surgery, radiation therapy, or any other treatment, in order to sensitize the tumor cells or to otherwise confer a therapeutic benefit to the patient. Additionally, the combination can be co-administered as neoadjuvant therapy following localized treatment (e.g., surgery, radiation, or both).

Further, the combination can be administered as a second line therapy, such as, but not limited to, once first line therapy has failed. Alternatively, the combination can be administered concurrently with first line therapy, and or at any point during first line therapy, which can be administered following initial treatment.

This is because a combination of an anti-CLTA-4 antibody and indolinone RTKI can provide a therapeutic benefit once first line therapy has failed, once systemic adjuvant therapy has failed, and the like. Thus, the invention encompasses administration of a antibody and indolinone RTKI in combination, with or without additional therapy, including, but not limited to, hormonal, radiotherapy, and any additional therapeutic agent (chemotherapy, signal inhibition therapy, among others), and the like, as would be appreciated by one skilled in the art based upon the disclosure provided herein.

The invention also relates to an article of manufacture (e.g., dosage form adapted for i.v. administration) comprising a human anti-CTLA4 antibody in the amount effective to treat cancer (e.g., at least 1 mg/kg, at least 3 mg/kg, at least 5 mg/kg, at least 10 mg/kg, at least 15 mg/kg, or at least 20 mg/kg) and a therapeutically effective amount of indolinone RTKI. In certain embodiments, the article of manufacture comprises a container or containers comprising a human anti-CTLA4 antibody, indolinone RTKI, and a label and/or instructions for use to treat cancer.

V. Pharmaceutical Compositions

The invention encompasses the preparation and use of pharmaceutical compositions comprising a human anti-CTLA4 antibody of the invention as an active ingredient in combination with and indolinone RTKI, e.g., compound 1, compound 2, and compound 3, among others. Such a pharmaceutical composition may consist of each active ingredient alone, as a combination of at least one active ingredient (e.g., an effective dose of an anti-CTLA4, an effective dose of indolinone RTKI) in a form suitable for administration to a subject, or the pharmaceutical composition may comprise the active ingredient and one or more pharmaceutically acceptable carriers, one or more additional (active and/or inactive) ingredients, or some combination of these.

In one embodiment, the antibody is administered parenterally (e.g., intravenously) in an aqueous solution while the indolinone RTKI (e.g., compound 1, compound 2, compound 3, and the like) is administered orally in pill/capsule form. Preferred formulations and dosage forms of the indolinone RTKI are described in US 2004/0229930 (WO 2004/024127), the disclosures of which are incorporated herein by reference in their entireties. However, the skilled artisan would understand, based upon the disclosure provided herein, that the invention is not limited to these, or any other, formulations, doses, routes of administration, and the like. Rather, the invention encompasses any formulation or method of administering an antibody in combination with a indolinone RTKI, including, but not limited to, administering each agent separately in a different formulation via a different route of administration (e.g., administering an anti-CTLA4 antibody i.v., while co-administering an indolinone RTKI (compound 1) orally, among many others. Thus, the following discussion describes various formulations for practicing the methods of the invention comprising administration of any anti-CTLA4 antibody in combination with an indolinone RTKI, but the invention is not limited to these formulations, but comprises any formulation as can be readily determined by one skilled in the art once armed with the teachings provided herein for use in the methods of the invention.

The antibodies employed in the invention can be incorporated into pharmaceutical compositions suitable for administration to a subject. Typically, the pharmaceutical composition comprises the antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, trehalose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The antibodies may be in a variety of forms. These include, for example, liquid, semi solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies can be administered by a variety of methods known in the art, including, without limitation, oral, parenteral, mucosal, by-inhalation, topical, buccal, nasal, and rectal. For many therapeutic applications, the preferred route/mode of administration is subcutaneous, intramuscular, intravenous or infusion. Non-needle injection may be employed, if desired. As will be appreciated by the skilled artisan, the route and/or mode of administration will vary depending upon the desired results.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of sensitivity in individuals.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the antibody is administered in an intravenous formulation as a sterile aqueous solution containing 5 or 10 mg/ml of antibody, with sodium acetate, polysorbate 80, and sodium chloride at a pH ranging from about 5 to 6. Preferably, the intravenous formulation is a sterile aqueous solution containing 5 or 10 mg/ml of antibody, with 20 mM sodium acetate, 0.2 mg/ml polysorbate 80, and 140 mM sodium chloride at pH 5.5.

In another embodiment of the invention, the antibody is administered in a sterile solution comprising 20 mM histidine buffer, pH 5.5, 84 mg/ml trehalose dihydrate, 0.2 mg/ml polysorbate 80, and 0.1 mg/ml disodium ethylenediaminetetraacetic acid dihydrate. In one aspect, the formulation is packaged in clear glass vials with a rubber stopper and an aluminum seal. In another aspect, the vial contains about 20 mg/ml of antibody with a nominal fill of about 400 mg per vial.

In one embodiment, part of the dose is administered by an intraveneous bolus and the rest by infusion of the antibody formulation. For example, a 0.01 mg/kg intravenous injection of the antibody may be given as a bolus, and the rest of a predetermined antibody dose may be administered by intravenous injection. A predetermined dose of the antibody may be administered, for example, over a period of an hour and a half to two hours to five hours.

With regard to an indolinone RTKI, the RTKI can be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics, anti-diarrheals, chemotherapeutic agents, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The anti-CTLA4/indolinone RTKI active ingredient combination of the invention can be administered to an animal, preferably a human. While the precise dosage administered of each active ingredient will vary depending upon any number of factors, including but not limited to, the type of animal and type of disease state being treated, the age of the animal and the route(s) of administration.

The anti-CTLA4 antibody may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The indolinone RTKI can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the indolinone RTKI itself, as well as the type and severity of the disease being treated, the type and age of the animal, etc.

The antibody and indolinone RTKI can be co-administered in that they can be administered separately, on different dates or at different times of the day, as well as simultaneously or on the same date. Co-administration thus encompasses any temporal combination of administration of the antibody and the indolinone RTKI such that administration of the two mediates a therapeutic benefit to the patient that is detectably greater than administration of either agent in the absence of the other.

An antibody-indolinone RTKI combination of the invention may be co-administered with numerous other compounds (antihormonal therapy agents, cytokines, chemotherapeutic and/or antiviral drugs, among many others). Alternatively, the compound(s) may be administered an hour, a day, a week, a month, or even more, in advance of the antibody-indolinone RTKI combination, or any permutation thereof. Further, the compound(s) may be administered an hour, a day, a week, or even more, after administration of radiation, stem cell transplant, or administration of any therapeutic agent (e.g., cytokine, chemotherapeutic compound, and the like), or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds, and the like. Several instructive examples demonstrating methods of co-administering an antibody-indolinone RTKI to treat cancer are provided, but the invention is not limited in any way to these examples, which merely serve to illustrate methods encompassed by the invention.

VI. Kits

The invention includes various kits for treatment of cancer, The kits comprise a therapeutically effective amount of a human anti-CTLA4 antibody of the invention and a therapeutically effective amount of at least one indolinone RTKI, preferably, compound 1, 2, and 3, or a pharmaceutically acceptable salt thereof, more preferably, compound 1, along with an applicator and instructional materials which describe use of the combination to perform the methods of the invention. Although exemplary kits are described below, the contents of other useful kits will be apparent to the skilled artisan in light of the present disclosure. Each of these kits is included within the invention.

The invention includes a kit for treatment of renal cell carcinoma in a patient in need thereof. The kit includes a human anti-CTLA4 antibody of the invention and at least one indolinone RTKI. The kit further comprises an applicator, including, but not limited to, a syringe, for administration of the components of the kit to a patient. Further, the kit comprises an instructional material setting forth the pertinent information for the use of the kit to treat breast cancer in the patient.

More preferably, the kit comprises at least one anti-CTLA4 antibody selected from 4.1.1, 4.8.1, 4.10.2, 4.13.1, 4.14.3, 6.1.1, ticilimumab, 11.6.1, 11.7.1, 12.3.1.1, 12.9.1.1, and ipilimumab, even more preferably, the antibody is 4.13.1, ticilimumab, and ipilimumab.

In one embodiment, the indolinone RTKI is compound 1.

The invention encompasses a kit comprising any combination of an anti-CTLA4 antibody and any indolinone RTKI, such as, but not limited to, compound 1. While such kit is preferred, the invention is not limited to this particular combination. Further, the kit can comprise a wide plethora of additional agents for treatment of cancer. Such agents are set forth previously and include chemotherapeutic compounds, cancer vaccines, signal transduction inhibitors other than an indolinone RTKI, agents useful in treating abnormal cell growth or cancer, antibodies or other ligands that inhibit tumor growth by binding to IGF-1R, a chemotherapeutic agent (taxane, vinca alkaloid, platinum compound, intercalating antibiotics, among many others), and cytokines, among many others, as well as palliative agents to treat, e.g., any toxicities that arise during treatment such as, but not limited to, an anti-diarrheal, an anti-emetic, and the like.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Anti-CTLA4 Antibody in Combination with Indolinone RTKI (Compound 1) for First-Line Treatment of Metastatic Renal Cell Carcinoma

Following surgery/radiotherapy, if any, patients having metastatic renal cell carcinoma (RCC) with at least one lesion that can be accurately measured in two dimensions and whose size is >2 cm×1 cm by conventional CT scan or >1 cm×1 cm by spiral CT scan are given standard chemotherapy using compound 1 (SU11248) per established protocols. Briefly, compound 1 is administered orally once per day at about 50 mg per day for four weeks. Following a two week resting period, a second four week course of compound 1 is administered to the patient. The cycle of compound 1 followed by rest is repeated as indicated.

The patient is further administered a single IV infusion (100 mL/hr) of anti-CTLA4 antibody as described herein at a dose of about 3 mg/kg, 6 mg/kg, 10 mg/kg or 15 mg/kg. Prophylactic anti-emetics and anti-diarrheals are given as appropriate. The treatment is repeated after 28 days without escalation of the anti-CTLA4 antibody dose, every 28 days thereafter for maximum of 12 cycles in the absence of intolerable toxicity or disease progression.

Preferably, the patient is premedicated with antihistamine (H1) at least one half hour prior to infusion of anti-CTLA4. Premedication is recommended but not required.

Compound 1 is administered sequentially or simultaneously with human anti-CTLA4 antibody ticilimumab, either once, or repeatedly, as determined.

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated, A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum 1g level), are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. Preferably, the antibody has the heavy and light chain amino acid sequences of ticilimumab.

### Example 2

### Anti-CTLA4 Antibody in Combination with Indolinone RTKI for First-Line Treatment of Metastatic Renal Cell Carcinoma

Following surgery/radiotherapy, if any, patients having metastatic renal cell carcinoma (RCC) with at least one lesion that can be accurately measured in two dimensions and whose size is >2 cm×1 cm by conventional CT scan or >1 cm×1 cm by spiral CT scan are given standard chemotherapy using an indolinone RTKI (e.g., compound 1, compound 2, or compound 3) per established protocols. Briefly, compound 1 is administered orally once per day at about 50 mg per day for a four week treatment period. Following a two week resting period, a second four week course of the compound is administered to the patient. The intermittent dosing cycle of treatment with the compound followed by a rest period is repeated as indicated.

The patient is further administered a single IV infusion (e.g., 100 mL/hr, 200 mL/hr, and the like) of an anti-CTLA4 antibody as described herein at a dose of about 3 mg/kg, 6 mg/kg, 10 mg/kg, or 15 mg/kg. Prophylactic anti-emetics and anti-diarrheals are given as appropriate. The antibody treatment is repeated after 28 days without escalation of the anti-CTLA4 antibody dose, every 28 days thereafter for maximum of 12 cycles in the absence of intolerable toxicity or disease progression.

Preferably, the patient is premedicated with antihistamine (H1) at least one half hour prior to infusion of anti-CTLA4. Premedication is recommended but not required.

Indolinone RTKI is administered prior to administration of the anti-CTLA4 antibody, either once, or repeatedly, as determined. The level of immune responsiveness of the patient is assessed prior to, during, and following each administration of indolinone RTKI (e.g., compound 1, compound 2, compound 3, etc.). The antibody is administered following the course of indolinone RTKI and after a detectable increase in the immune responsiveness of the patient if a decrease in immune responsiveness is detected upon administration of indolinone RTKI. Generally, the antibody is administered about 1 to 100 days after the last dose of indolinone RTKI is administered to the patient.

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

Preferably, the anti-CTLA4 antibody has the heavy and light chain amino acid sequences of at least one antibody selected from 4.1.1, 4.13.1, ticilimumab, and ipilimumab. Preferably, the antibody has the heavy and light chain amino acid sequences of ticilimumab.

### Example 3

### Anti-CTLA4 Antibody in Combination with Indolinone RTKI Treatment of Imatinib-Resistant Gastrointestinal Stromal Tumor (GIST)

Following surgery/radiotherapy, if any, patients having imatinib-resistant GIST are given standard chemotherapy using indolinone RTKI (compound 1) per established protocols. Briefly, compound 1 is administered orally once per day at about 50 mg per day for four weeks. Following a two week resting period, a second four week course of compound 1 is administered to the patient. The cycle of treatment with compound 1 followed by a resting period is repeated as indicated.

The patient is further administered a single IV infusion (100 mL/hr) of an anti-CTLA4 antibody as described herein at a dose of about 1 mg/kg, 3 mg/kg, 6 mg/kg, 10 mg/kg, or 15 mg/kg. Prophylactic anti-emetics and anti-diarrheals are given as appropriate. The treatment is repeated after 28 days without dose escalation of the anti-CTLA4 antibody dose, every 28 days thereafter for maximum of 12 cycles in the absence of intolerable toxicity or disease progression.

Preferably, the patient is premedicated with antihistamine (H1) at least one half hour prior to infusion of anti-CTLA4. Premedication is recommended but not required.

Compound 1 is administered sequentially or simultaneously with anti-CTLA4 antibody either once, or repeatedly, as determined.

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. Preferably, the antibody has the heavy and light chain amino acid sequences of ticilimumab.

### Example 4

### Anti-CTLA4 Antibody in Combination with Indolinone RTKI (Compound 1) for First-Line Treatment of Metastatic Renal Cell Carcinoma

Following surgery/radiotherapy, if any, patients having metastatic renal cell carcinoma (RCC) with at least one lesion that can be accurately measured in two dimensions and whose size is ≧2 cm×1 cm by conventional CT scan or ≧1 cm×1 cm by spiral CT scan are given standard chemotherapy using compound 1 (sunitinib malate, SUTENT, SU11248) per established protocols. Briefly, compound 1 is administered orally once per day at about 37.5 mg every day and administration is continuous (i.e., without a resting period).

The patient is further administered a single IV infusion (100 mL/hr) of anti-CTLA4 antibody as described herein at a dose of about 10 mg/kg or 15 mg/kg. Prophylactic anti-emetics and anti-diarrheals are given as appropriate. The treatment is repeated after three months without escalation of the anti-CTLA4 antibody dose, and every three months thereafter for maximum of 12 cycles in the absence of intolerable toxicity or disease progression.

Preferably, the patient is premedicated with antihistamine (H1) at least one half hour prior to infusion of anti-CTLA4. Premedication is recommended but not required.

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. Preferably, the antibody has the heavy and light chain amino acid sequences of ticilimumab.

### Example 5

### Anti-CTLA4 Antibody in Combination with Indolinone RTKI Treatment of Imatinib-Resistant Gastrointestinal Stromal Tumor (GIST)

Following surgery/radiotherapy, if any, patients having imatinib-resistant GIST are given standard chemotherapy using indolinone RTKI (compound 1) per established protocols. Briefly, compound 1 is administered orally once per day at about 37.5 mg per day continuously.

The patient is further administered a single IV infusion (100 mL/hr) of an anti-CTLA4 antibody as described herein at a dose of about 10 mg/kg, or 15 mg/kg. Prophylactic anti-emetics and anti-diarrheals are given as appropriate. The antibody treatment is repeated after three months without dose escalation of the anti-CTLA4 antibody dose, and every three months thereafter for maximum of 12 cycles in the absence of intolerable toxicity or disease progression.

Preferably, the patient is premedicated with antihistamine (H1) at least one half hour prior to infusion of anti-CTLA4. Premedication is recommended but not required.

Ticilimumab is provided in 20 ml clear glass vials with a rubber stopper and an aluminum seal. Each vial contains 20 mg/ml (with a nominal fill of 400 mg/vial) of ticilimumab, in a sterile aqueous solution comprising 20 mM histidine buffer, pH 5.5, 84 mg/ml trehalose dihydrate, 0.2 mg/ml polysorbate 80, and 0.1 mg/ml disodium EDTA dihydrate.

For all patients, ECOG performance status, vital signs, and body weight are assessed pre-dose, and vital signs can be repeated post-dose, as clinically indicated. A physical examination (including opthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

The anti-CTLA4 antibody has the heavy and light chain amino acid sequences of at least one antibody selected from 4.1.1, 4.13.1, ticilimumab, and ipilimumab. Preferably, the antibody has the heavy and light chain amino acid sequences of ticilimumab.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for the treatment of cancer in a patient in need of such treatment, said method comprising administering to said patient a therapeutically effective amount of an anti-CTLA4 antibody, or antigen-binding portion thereof, in combination with a therapeutically effective amount of an indolinone receptor tyrosine kinase inhibitor (RTKI).

2. The method of claim 1, wherein said indolinone RTKI is at least one compound selected from the group consisting of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide, N-[2-(ethylamino)ethyl]-5-[(Z)-(5-fluoro-2-oxo-1,2-dihydro-3H-indol-3-ylidene)methyl]-2,4-dimethyl-1H-pyrrole-3-carboxamide, and 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, or a pharmaceutically acceptable salt thereof.

3. The method of claim 2, wherein said RTKI is N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide.

4. The method of claim 3, wherein said therapeutically effective amount of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide ranges from about 25 mg to 87.5 mg per day, and wherein said N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide is administered orally.

5. The method of claim 4, wherein said therapeutically effective amount of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide ranges from about 37.5 mg to 50 mg per day.

6. The method of claim 3, wherein said N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide is administered according to a dosing regimen selected from the group consisting of administration every day, administration every day for approximately four weeks, administration every day for approximately four weeks followed by a resting period of about two weeks, administration every day for approximately three weeks followed by a resting period of about one week, administration every day for approximately two weeks followed by a resting period of about one week.

7. The method of claim 1, wherein said treatment is a therapy selected from the group consisting of neoadjuvant therapy, adjuvant therapy, first-line therapy and second-line therapy.

8. The method of claim 1, wherein said therapeutically effective amount of said human anti-CTLA4 antibody ranges from about 0.1 mg/kg to 50 mg/kg.

9. The method of claim 8, wherein said therapeutically effective amount of said human anti-CTLA4 antibody ranges from about 0.3 mg/kg to 20 mg/kg.

10. The method of claim 9, wherein said therapeutically effective amount of said human anti-CTLA4 antibody is selected from the group consisting of at least 1 mg/kg, at least 3 mg/kg, at least 6 mg/kg, at least 10 mg/kg, and at least 15 mg/kg.

11. The method of claim 10, wherein said antibody is administered according to a dosing regimen selected from the group consisting of administration of about 6 mg/kg every twenty-eight days, administration of about 6 mg/kg every three months, administration of about 10 mg/kg every twenty-eight days, administration of about 10 mg/kg every three months, administration of about 15 mg/kg every twenty-eight days, and administration of about 15 mg/kg every three months.

12. The method of claim 5, wherein said N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide is administered at about 37.5 mg per day and said anti-CTLA4 antibody is administered according to a dosing regimen selected from the group consisting of administration of about 10 mg/kg every three months and administration of about 15 mg/kg every three months.

13. The method of claim 1, wherein said cancer is selected from the group consisting of breast cancer, prostate cancer, ovarian cancer, pancreatic cancer, lung cancer, acute myeloid leukemia, colorectal carcinoma, renal cell carcinoma, gastrointestinal stromal tumor, and sarcoma.

14. The method of claim 1, wherein said anti-CTLA4 antibody, or antigen-binding portion thereof, is at least one antibody selected from the group consisting of:
(a) a human antibody having a binding affinity for CTLA4 of about 10⁻⁸ or greater, and which inhibits binding between CTLA4 and B7-1, and binding between CTLA4 and B7-2;
(b) a human antibody having an amino acid sequence comprising at least one human CDR sequence that corresponds to a CDR sequence from an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab;
(c) a human antibody having the amino acid sequences of the heavy and light chains of an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab;
(d) an antibody, or antigen-binding portion thereof, that competes for binding with CTLA4 with at least one antibody having the amino acid sequences of the heavy and light chains of an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab; and
(e) an antibody, or antigen-binding portion thereof, that cross-competes for binding with CTLA4 with at least one antibody having the amino acid sequences of the heavy and light chains of an antibody selected from the group consisting of 4.1.1, 4.8.1, 4.10.2, 4.13.1, 4.14.3, 6.1.1, ticilimumab, 11.6.1, 11.7.1, 12.3.1.1, 12.9.1.1, and ipilimumab.

15. The method of claim 14, where said antibody is a human antibody having the amino acid sequences of the heavy and light chains of an antibody selected from the group consisting of 4.1.1, 4.13.1, ticilimumab and ipilimumab.

16. The method of claim 15, wherein said antibody is ticilimumab.

17. The method of claim 14, wherein said antibody, or antigen-binding portion thereof, comprises a heavy chain and a light chain wherein the amino acid sequences of the heavy chain variable domain of said heavy chain and the light chain variable domain of said light chain are selected from the group consisting of:
(a) the amino acid sequence of SEQ ID NO:3 and the amino acid sequence of SEQ ID NO:9;
(b) the amino acid sequence of SEQ ID NO:15 and the amino acid sequence of SEQ ID NO:21;
(c) the amino acid sequence of SEQ ID NO:27 and the amino acid sequence of SEQ ID NO:33;
(d) the amino acid sequence encoded by the nucleic acid sequence of SEQ ID NO:1 and the amino acid sequence encoded by the nucleic acid sequence of SEQ ID NO:7;
(e) the amino acid sequence encoded by the nucleic acid sequence of SEQ ID NO:13 and the amino acid sequence encoded by the nucleic acid sequence of SEQ ID NO:19;
(f) the amino acid sequence encoded by the nucleic acid sequence of SEQ ID NO:25 and the amino acid sequence encoded by the nucleic acid sequence of SEQ ID NO:31; and
(g) the amino acid sequence of ipilimumab.

18. The method of claim 14, wherein said antibody, or antigen-binding portion thereof, is an antibody selected from the group consisting of:
(a) an antibody comprising the amino acid sequences set forth in SEQ ID NO:4, SEQ ID NO:5, SEQ ID NO:6, SEQ ID NO:10, SEQ ID NO:11 and SEQ ID NO:12;
(b) an antibody comprising the amino acid sequences set forth in SEQ ID NO:16, SEQ ID NO:17, SEQ ID NO:18, SEQ ID NO:22, SEQ ID NO:23 and SEQ ID NO:24; and
(c) an antibody comprising the amino acid sequences set forth in SEQ ID NO:28, SEQ ID NO:29, SEQ ID NO:30, SEQ ID NO:34, SEQ ID NO:35 and SEQ ID NO:36.

19. The method of claim 14, wherein said antibody, or antigen-binding portion thereof, comprises a heavy chain variable region having the amino acid sequence set forth in SEQ ID NO:27 and a light chain variable region having the amino acid sequence set forth in SEQ ID NO:33.

20. The method of claim 14, wherein said antibody is selected from the group consisting of:
(a) an antibody comprising the amino acid sequences set forth in SEQ ID NO:2 and SEQ ID NO:8;
(b) an antibody comprising the amino acid sequences set forth in SEQ ID NO:14 and SEQ ID NO:20; and
(c) an antibody comprising the amino acid sequences set forth in SEQ ID NO:26 and SEQ ID NO:32.

21. The method of claim 1, wherein said antibody is administered after completion of a course of said indolinone RTKI and wherein the level of immune response in said patient is greater than the level of immune response in said patient during or immediately after said course of indolinone RTKI.

22. The method of claim 21, wherein said antibody is administered from about one to one-hundred days after said course of indolinone RTKI.

23. A pharmaceutical composition for treatment of cancer, said composition comprising a therapeutically effective amount of an anti-CTLA4 antibody, or antigen-binding portion thereof, and a therapeutically effective amount of an indolinone RTKI, and a pharmaceutically acceptable carrier.

24. The pharmaceutical composition of claim 23, wherein said indolinone RTKI is at least one compound selected from the group consisting of N-[2-diethylamino]ethyl]-5-[(Z)-(5-fluoro-2-oxo-1,2-dihydro-3H-indol-3-ylidene)methyl]-2,4-dimethyl-1H-pyrrole-3-carboxamide, N-[2-(ethylamino)ethyl]-5-[(Z)-(5-fluoro-2-oxo-1,2-dihydro-3H-indol-3-ylidene)methyl]-2,4-dimethyl-1H-pyrrole-3-carboxamide, and 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, or a pharmaceutically acceptable salt thereof.


SEQUENCE LISTING (36 sequences)

SEQ ID NO: 1 - DNA, 1392 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 444 aa, Homo sapiens
QVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 118 aa, Homo sapiens
QVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSS

SEQ ID NO: 4 - protein, 10 aa, Homo sapiens
GFTFSSHGMH

SEQ ID NO: 5 - protein, 15 aa, Homo sapiens
VIWYDGRNKYYADSV

SEQ ID NO: 6 - protein, 9 aa, Homo sapiens
GGHFGPFDY

SEQ ID NO: 7 - DNA, 708 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtattagcagcagcttcttagcctggtaccagcagagacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 215 aa, Homo sapiens
EIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 108 aa, Homo sapiens
EIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIK

SEQ ID NO: 10 - protein, 12 aa, Homo sapiens
RASQSISSSFLA

SEQ ID NO: 11 - protein, 7 aa, Homo sapiens
GASSRAT

SEQ ID NO: 12 - protein, 9 aa, Homo sapiens
QQYGTSPWT

SEQ ID NO: 13 - DNA, 1335 bp, Homo sapiens
caggtgcagctggtggagtctgggggaggcgtggtccagcctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 444 aa, Homo sapiens
QVQLVESGGGVVQPGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 118 aa, Homo sapiens
QVQLVESGGGVVQPGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSS

SEQ ID NO: 16 - protein, 10 aa, Homo sapiens
GFTFSSHGIH

SEQ ID NO: 17 - protein, 15 aa, Homo sapiens
VIWYDGRNKDYADSV

SEQ ID NO: 18 - protein, 9 aa, Homo sapiens
VAPLGPLDY

SEQ ID NO: 19 - DNA, 645 bp, Homo sapiens
gaaattgtgttgacgcagtctccaggcaccctgtctttgtctccaggggaaagagccaccctctcctgcagggccagtcagagtgtcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcaacagtatggtaggtcaccattcactttcggccctgggaccaaagtagatatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, Homo sapiens
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 107 aa, Homo sapiens
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIK

SEQ ID NO: 22 - protein, 11 aa, Homo sapiens
RASQSVSSYLA

SEQ ID NO: 23 - protein, 7 aa, Homo sapiens
GASSRAT

SEQ ID NO: 24 - protein, 9 aa, Homo sapiens
QQYGRSPFT

SEQ ID NO: 25 - DNA, 1413 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 125 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 28 - protein, 10 aa, Homo sapiens
GFTFSSYGMH

SEQ ID NO: 29 - protein, 15 aa, Homo sapiens
VIWYDGSNKYYADSV

SEQ ID NO: 30 - protein, 16 aa, Homo sapiens
DPRGATLYYYYYGMDV

SEQ ID NO: 31 - DNA, 714 bp, Homo sapiens
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 32 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 107 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 34 - protein, 11 aa, Homo sapiens
RASQSINSYLD

SEQ ID NO: 35 - protein, 7 aa, Homo sapiens
AASSLQS

SEQ ID NO: 36 - protein, 9 aa, Homo sapiens
QQYYSTPFT